(19) United States
(12) Patent Application Publication
(10) Pub. No.: US 2019/0218292 A1
(43) Pub. Date: Jul. 18, 2019

(54) ANTIBODY FOR CANCER TREATMENT

(71) Applicant: LABORATOIRE FRANÇAIS DU FRACTIONNEMENT ET DES BIOTECHNOLOGIES, Les Ulis (FR)

(72) Inventor: Céline MONNET, Lambersart (FR)

(73) Assignee: LABORATOIRE FRANÇAIS DU FRACTIONNEMENT ET DES BIOT ECHNOLOGIES, Les Ulis (FR)

(21) Appl. No.: 16/305,832

(22) PCT Filed: May 31, 2017
(86) PCT No.: PCT/EP2017/063158

§ 371 Date: Nov. 29, 2018

(30) Foreign Application Priority Data
May 31, 2016 (FR) 1654891

(51) Int. Cl. C07K 16/28; A61P 35/00

(52) U.S. Cl. CPC C07K 16/2818; A61P 35/00; A61K 2039/505; C07K 2317/76; C07K 2317/52

(57) ABSTRACT

The present invention relates to an antibody inhibiting at least one immune checkpoint, and having a modified Fc region compared with that of the parent antibody.

The present invention is directed towards cancer immunotherapy.

### TECHNOLOGICAL BACKGROUND OF THE INVENTION

Immunotherapy via the administration of exogenous antibodies to patients is at present widely employed to treat various pathologies, and cancers in particular.

In recent years, knowledge of cancer biology and immunology has never ceased to progress. It is henceforth known that the immune system is involved in anti-tumoral response in particular via recognition of cancer cells by immune cells. This recognition may result in the controlling and even elimination of tumours. However, the immune effector cells have receptors on their surface known as immune checkpoints. The role of these receptors is to modulate (inhibit or activate) immune response, in particular to maintain self-tolerance. It is currently known that cancer cells follow this escape mechanism to evade the immune system, in particular through the expression on their surface of ligands of said immune checkpoint receptors, which will lead to inhibition of the response of the immune effector cells when recognized by the latter (Pardoll et al., Nat. Rev. Cancer. 12:252-264 (2012)). A new immunotherapy approach to cancers has thus seen the light to counter-attack this phenomenon of immune escape, by restoring immune response and hence tumour rejection. Therefore, over the last few years antibodies directed against these immune checkpoints have been developed. The chief antibodies on the market or under development are:
Cytotoxic T-Lymphocyte Associated antigen 4 (CTLA4) (e.g. Ipilimumab—Yervoy® Bristol Myers Squibb). This monoclonal antibody causes blocking of the CTLA4 receptor, an inhibitory immune checkpoint present on T-lymphocytes. and therefore leads to activation of the immune response of said T-lymphocyte. In other words, Ipilimumab blocks the immune escape pathway of cancer cells by suppressing their inhibitory action on T-lymphocytes via their stimulation of the CTLA4 receptor. A clinical trial for the first time evidenced that patients suffering from metastatic melanomas treated with an anti-CTLA4 antibody (Ipilimumab), had an increased lifetime (Hodi et al., N Engl. J. Med., 363:711-723 (2010) and 363:1290 (2010) (erratum); Robert et al., N Engl. J. Med., 364:2517-2526 (2011));
Programmed Death Cell 1 (PD1) (e.g. Nivolumab—Opdivo® Bristol Myers Squibb and Pembrolizumab—Keytruda® Merck). This monoclonal antibody causes blocking of the PDI receptor, an inhibitory immune checkpoint present on T-lymphocytes, and therefore leads to activation of the immune response of said T-lymphocyte. In other words, Nivolumab blocks the immune escape pathway of cancer cells by suppressing their inhibitory action on T-lymphocytes via their stimulation of the PD-1 receptor. Nivolumab notably appears to have anti-tumoral activity in patients suffering from metastatic renal cell carcinoma (Motzer et al., American Society of Clinical Oncology 33 (13): 1430-1437 (2014)); or
the Activation Gene 3 (LAG3) lymphocyte (e.g. BMS-986016). LAG3 is an inhibitory immune checkpoint present on T-lymphocytes. It is presently the focus of inhibitor development and in particular of monoclonal antibodies. Similar to CTLA4 and PD1, the blockading of this receptor would allow blocking of its inhibitor effect on the response of effector cells, and thereby activate immune response.

There is a current need to optimize these antibodies directed against an immune checkpoint, in particular to obtain a more efficient or less toxic clinical effect. In particular, there is a need to obtain antibodies having an improved half-life in the body allowing extended anti-tumoral effect, whilst being well tolerated by the body and/or advantageously having improved effector properties.

### SUMMARY OF THE INVENTION

The present invention therefore relates to an antibody, particularly an inhibitory antibody, targeted against at least one immune checkpoint, having a modified Fc region compared with that of a parent antibody. Said antibody is adapted for use in the treatment of cancer. Preferably, the antibody of the invention has modified affinity for at least one of the Fc receptors (FcRs) compared with the parent Fc region.

The invention also relates to a pharmaceutical composition comprising at least one antibody of the invention. Said composition is suitable for use in the treatment of cancers. Preferably, the antibody of the invention has an extended half-life and/or modified, especially decreased, functional activity.

### KEY TO FIGURES

FIG. 1 shows the aligning of native human IgG1 sequences relating to positions 216 to 447 (according to EU numbering) with the corresponding human IgG2 (SEQ ID NO: 7), human IgG3 (SEQ ID NO: 8) and human IgG4 (SEQ ID NO: 9) sequences. The IgG1 sequences refer to the G1 m1,17 allotype (SEQ ID NO: 6) and G1m3 allotype (SEQ ID NO: 10). The “CH2-CH3 lower hinge” region of IgG1 starts at cysteine 226 (see arrow). The CH2 region is highlighted in grey and the CH3 region is in italics.

FIG. 2 shows the results of binding to human FcRn (hFcRn) on cells by various nivolumab mutants, and FIG. 3 shows the results of binding to human CD16aV (hCD16aV) on cells by various nivolumab mutants.

The sequences are the following:

| SEQ ID NO: | Sequences |
|---|---|
| 1 | Human IgG1 G1m,17 Fc (residues 226-447 |
| | as per EU numbering) |
| 2 | Human IgG2 Fc (residues 226-447 as per |
| | EU numbering) |
| 3 | Human IgG3 Fc (residues 226-447 as per |
| | EU numbering) |
| 4 | Human IgG4 Fc (residues 226-447 as per |
| | EU numbering) |
| 5 | Human IgG1 G1m3 Fc (residues 226-447 |
| | as per EU numbering) |
| 6 | Human IgG1 G1m1,17 Fc (residues 216-447 |
| | as per EU numbering) |
| 7 | Human IgG2 Fc |
| 8 | Human IgG3 Fc |
| 9 | Human IgG4 Fc |
| 10 | Human IgG1 G1m3 Fc |
| 11 | nivolumab VH |
| 12 | nivolumab VL |
| 13 | pembrolizumab VH |
| 14 | pembrolizumab VL |

### DETAILED DESCRIPTION

Characteristics of the Antibodies

The present invention relates to an antibody directed against an immune checkpoint, having a modified Fc region compared with that of a parent antibody.

More particularly, the present invention relates to an antibody inhibiting at least one immune checkpoint and having a modified Fc region compared with that of a parent antibody.

By a «antibody» is meant a tetramer formed of two heavy chains, each of 50-70 kDa (called H chains) and of two light chains of about 25 kDa each (called L chains) linked by identical intra- and interchain disulfide bridges. This tetramer comprises at least two variable regions at the N-terminal end of each chain (called VL for the light chains and VH for the heavy chains) and a constant region at the C-terminal end formed of a single domain called CL for light chain and of three or four domains for the heavy chain called CH1, CH2, CH3 and when applicable CH4.

Each domain comprises about 110 amino acids and is of comparable structure. The 2 heavy chains are linked by disulfide brides at the CH2s and each heavy chain is linked to a light chain via a disulfide bridge between CH1 and CL. The region which determines the specificity of the antibody for the antigen is carried by the variable portions, it is these portions that are responsible for recognition of the antigen. In each variable domain, three loops are grouped together to form a binding site to the antigen. Each of the loops is called a Complementarity-Determining Region (or CDR). Regarding the constant portions of the heavy chains which form the Fc region, these can bind to the Fc receptors (FcR) of the effector cells or of molecules such as complement proteins to induce different functional properties. The assembling of the chains which compose an antibody allows the defining of a characteristic Y-shaped three-dimensional structure in which:
the base of the Y corresponds to the constant region Fc, or Fc fragment: it is recognized by complement proteins and the Fc receptors to mediate the effector functions of the antibody, and
the ends of the Y arms correspond to the respective assembling of the variable domain of a light chain and the variable domain of a heavy chain, said ends forming the Fab region and determining the specificity of the antibody for the antigen.

There are five types of heavy chains (alpha, gamma, delta, epsilon, mu), which determine the classes of immunoglobulins (IgA. IgG, IgD, IgE, IgM). The light chain group comprises two sub-types, lambda and kappa. The kappa and lambda light chains are shared by all classes and sub-classes. In man, the proportion of kappa and lambda produced is within a ratio of 2 to 1.

IgGs are the most abundant immunoglobulins of the antibodies circulating in the serum (75-80%). They are present in the form of monomers and have a half-life of 21 days (longer than the other immunoglobulins).

There are four types of gamma heavy chains, determining four IgG sub-classes (IgG1 for gamma1, IgG2 for gamma2. IgG3 for gamma3 and IgG4 for gamma4). These four sub-classes differ in the number and variable positions of the disulfide bridges (Basic and Clinical Immunology, 8^{th} Edition, Daniel P. Stites, Abba I. Terr and Tristram G. Parslow (Eds.), Appleton & Lange, Norwalk, Conn., 1994, page 71 and Chapter 6).

The four sub-classes of human IgGs also differ in respect of biological activity despite their most homologous structures (more than 95% sequence homology for the Fc regions).

Antibodies particularly comprise full-length immunoglobulins, monoclonal antibodies, multi-specific antibodies, chimeric antibodies, humanized antibodies and fully human antibodies.

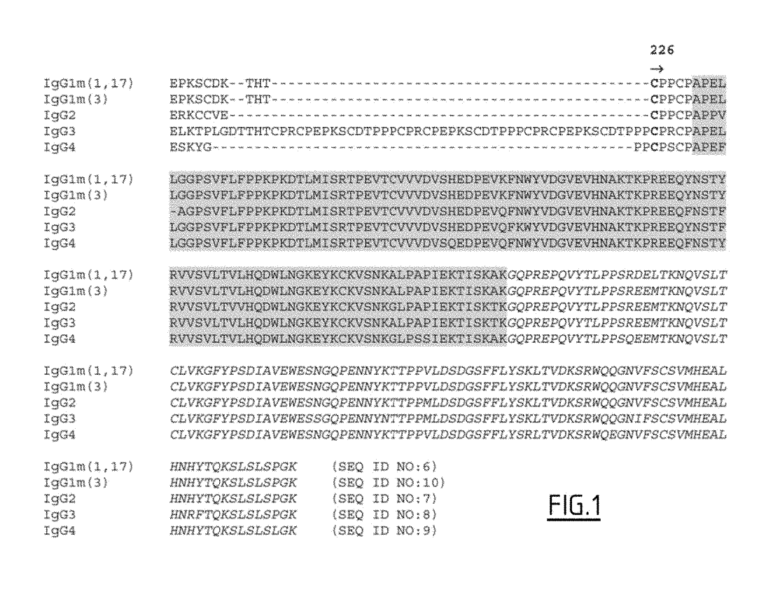

The term “Fc” or “Fc region” or “Fc fragment” designates the constant region of the heavy chain of an antibody with the exception of the first domain of the immunoglobulin constant region (CH1). Therefore, Fc refers to the two last domains (CH2 and CH3) of an IgG constant region and to the flexible N-terminal hinge of these domains. For a human IgG1, the Fc region comprises the domains CH2 and CH3 and the lower hinge region between CH1 and CH2. Therefore, the Fc region corresponds to residue C226 as far as its carboxy-terminal end, i.e. the residues at position 226 to 447 as per EU numbering or the Kabat equivalent. The analogue domains for other IgG sub-classes can be determined by aligning the amino acid sequences of the heavy chains or heavy chain fragments of the IgG sub-classes with human IgG1 (Cf. FIG. 1). The Fc region used may also comprise a portion of the upper hinge region located between positions 216 to 226 as per EU numbering or the Kabat equivalent; in this case, the Fc region used corresponds to the residues at position 216 to 447, 217 to 447, 218 to 447, 219 to 447, 220 to 447, 221 to 447, 222 to 447, 223 to 447, 224 to 447 or 225 to 447, as per EU numbering or the Kabat equivalent. Preferably in this case the Fc region used corresponds to the residues at position 216 to 447, as per EU numbering or the Kabat equivalent.

Preferably, the Fc region used is selected from among the sequences SEQ ID NO: 1, 2, 3, 4 and 5. Preferably, the Fc region of the parent antibody is an IgG1. Preferably, the Fc region of the parent antibody has the sequence SEQ ID NO: 1. The sequences represented in SEQ ID NO: 1, 2, 3, 4 and 5 are devoid of an N-terminal hinge region.

The sequences represented in SEQ ID NO: 6, 7, 8, 9 and 10 respectively correspond to the sequences represented in SEQ ID NO: 1, 2, 3, 4 and 5 with their N-terminal hinge regions. Therefore, in one particular embodiment, the Fc region of the parent antibody is selected from among the sequences SEQ ID NO: 6, 7, 8, 9 and 10.

Preferably, the Fc region of the parent antibody has a sequence corresponding to the positions 1-232, 2-232, 3-232, 4-232, 5-232, 6-232, 7-232, 8-232, 9-232, 10-232 or 11-232 of sequence SEQ ID NO: 6.

Preferably, and alternatively, the Fc region of the parent antibody is an IgG4. Preferably, the sequence of the Fc region of the parent antibody is SEQ ID NO: 4 or 9. Further preferably, the Fc region of the parent antibody is an IgG4 mutated at position 228, preferably comprising the mutation S228P. Preferably, the Fc region of the parent antibody has the sequence SEQ ID NO: 4 or 9, more preferably comprising the mutation S228P.

By «position» is meant a position in the sequence of amino acids. For the Fc region, the positions are numbered in accordance with EU numbering or the Kabat equivalent.

By «amino acid» or «residue» is meant one of the 20 natural amino acids or natural analogues.

The terms «immune checkpoints.» refer to receptors located on the surface of immune effector cells capable of inhibiting (immune checkpoint inhibitors) or stimulating (immune checkpoint activators) immune response after engagement thereof with their ligands.

By «activator receptor» in the present invention is meant a surface receptor which, after interaction with its ligand, causes triggering of a signalling pathway leading to activation of immune response. The immune checkpoint activator is preferably selected from among GITR and OX40.

Preferably, the target immune checkpoint of the antibodies of the invention is an inhibitory receptor of immune effector cells.

By a inhibitory receptor in the present invention is meant a surface receptor which, after interaction with its ligand, causes triggering of a signalling pathway leading to inactivation of immune response.

Preferably, the immune checkpoint inhibitor is selected from among PD1, CTLA4, TIM3, LAG3, KIR, BTLA1 and a2AR. PD1, CTLA4, TIM3, LAG3, KIR, BTLA1 and a2AR are inhibitory receptors of the immune effector cells. Preferably, the immune checkpoint inhibitor is PD1. Preferably, the antibody of the invention is an anti-PD1 comprising, as VH and VL, the sequences SEQ ID NO: 11 and 12 respectively, or else 13 and 14 respectively.

PD1 (Programmed cell Death factor 1) is an inhibitory receptor of the CD28 family expressed on the surface of activated T and B lymphocytes and Natural Killers. Its role is to limit the activity of effector cells in secondary lymphoid tissue or tumours, thereby imparting thereto a mechanism of major tumour resistance. PD1 inhibits lymphocyte functions when it is engaged with one of its ligands (PDL1 (or B7-H1 or CD274) and PDL2). PDL1 is a molecule expressed on the surface of the tumour cells and myeloid cells. In the event of chronic exposure to the PDL1 ligand (e.g. in cancers), the lymphocytes carrying PD1 on their surface have their function inhibited, leading to an anergy phenomenon. Anti-PD1 antibodies are used in the treatment of cancers such as lung cancer, non-small cell lung cancer (NSCLC), mesothelioma, bladder cancer, colorectal cancer, metastatic colorectal cancer, bladder cancer, breast cancer, head and neck cancer, testicular cancer, endometrial cancer, oesophageal cancer, thymus cancer, hematologic cancer, advanced hematologic cancer such as non-Hodgkin lymphomas, Hodgkin lymphomas, chronic lymphocytic leukaemia, multiple melanomas, acute myeloid leukaemia, brain tumours, glioblastomas, solid tumours, gastric adenocarcinomas, germ cell tumours, hepatocellular carcinomas, melanomas, metastatic melanomas, lymphomas, diffuse large B-cell lymphoma (DLBCL), follicular lymphomas, non-resectable or metastatic melanomas, or advanced renal cell carcinomas.

CTLA4 (Cytotoxic T-lymphocyte-associated antigen 4) is an inhibitory receptor expressed solely on the surface of T-lymphocytes. Its role is to 6 regulate the first activation steps of T-lymphocytes. 48 hours after activation of T-lymphocytes T via their receptor (T Cell Receptor—TCR), CTLA4 engages with its ligands (CD80 or CD86) which are expressed on the surface of antigen presenting cells (APCs) at the lymph nodes and sometimes at tumours. This causes a signalling cascade leading to inhibition of the T-lymphocytes. Anti-CTLA4 antibodies are used in the treatment of cancers such as lung cancer, non-small cell lung cancer (NSCLC), small cell lung cancer, breast cancer, pancreatic cancer, prostate cancer, gastric cancer, renal cancer, head and neck cancer, liver cancer, metastatic or non-resectable melanomas, skin melanomas with lymph node involvement, renal carcinomas, myelomas, lymphomas, hepatocellular carcinomas, brain metastases, solid tumours, mesothelioma, lymphomas or melanomas.

TIM3 (T-cell immunoglobulin and mucin-domain containing-3) is a receptor expressed on the surface of IFN γ-secreting T lymphocytes. One of the ligands thereof is galectine-9, which is a protein over-expressed in tumour cells. The engaging of TIM3 with galectine-9 leads to inhibition of immune response.

LAG3 (lymphocyte activation gene 3) also called CD223 is a molecule expressed on the surface of T-lymphocytes. Its only known ligand is the type II Major Histocompatibility Complex (MHC II) which can be expressed by some cancer cells but also by antigen presenting cells (APCs—macrophages and dendritic cells) infiltrated in tumours. The engaging of LAG3 with its receptor leads to an anergy phenomenon.

KIR (Killer-cell immunoglobulin-like receptor) is a molecule expressed on the surface of Natural Killers, T-lymphocytes and APCs. When KIR binds to its ligand, type I MHC (MHC I), the effector response of Natural Killers is attenuated in tumours.

BTLA1 (B- and T-lymphocyte attenuator), also called CD272, is a molecule expressed on the surface of lymphocytes. Its ligand, the HVEM molecule (herpesvirus entry mediator), is expressed in certain types of tumours (in particular in melanomas).

a2ARs are expressed in different types of immune effector cells, in particular in T-lymphocytes, and in endothelial cells. When a2AR binds to its ligand—adenosine (which accumulates in tumours), the CD4+ cells express FOXP3 and therefore differentiate into regulatory T cells, the result of which is to inhibit immune response.

By «inhibitory antibody» is meant an antibody capable of binding to its target immune checkpoint inhibitor to prevent binding thereof with its endogenous ligand. In the context of the present invention, an inhibitory antibody of at least one immune checkpoint inhibitor is capable of binding to said checkpoint to neutralize the latter and hence prevent binding thereof with its ligand thereby blocking the inhibitory signalling pathways of immune response following from this interaction.

By «activator antibody» a is meant an antibody capable of binding to its target immune checkpoint activator and hence of stimulating the activator signalling pathways of immune response following from this interaction. By a activator antibody is also meant an antibody capable of binding to its target which is an endogenous ligand inhibiting an immune checkpoint activator expressed on the surface of cancer cells. By binding to the endogenous inhibitor of an immune checkpoint activator expressed on the surface of cancer cells, said antibody prevents binding thereof with its receptor and thereby blocks inhibition of immune response following from this interaction.

The term «parent antibody» is used to define the reference antibody which can be of natural or synthetic origin. In the context of the present invention, the parent antibody comprises an Fc region called a «parent Fc region». Said parent Fc region is selected from the group of wild-type Fc regions and their fragments. By “wild-type or WT” is meant here a sequence of amino acids or a nucleotide sequence found in nature i.e. is of natural origin including allelic variations, and which has not been intentionally modified using molecular biology techniques such as mutagenesis. For example, «wild-type» regions notably refer to the Fc region of IgG1 having the sequence SEQ ID NO: 1 (G1m1,17 allotype), the Fc region of IgG2 having the sequence SEQ ID NO: 2, the Fc region of IgG3 having the sequence SEQ ID NO: 3, the Fc region of IgG4 having the sequence SEQ ID NO: 4, and the Fc region of IgG1 having the sequence SEQ ID NO:5 (G1m3 allotype). The Fc regions of «wild-type» also refer to the Fc regions corresponding to sequences SEQ ID NO: 6 to SEQ ID NO: 10. Preferably, the parent antibody comprises a human Fc region, preferably an Fc region of human IgG1 or human IgG4. The parent antibody may comprise modifications of amino acids pre-existing in the Fc region (e.g. an Fc mutant) compared with Fc regions of wild-type.

By «immune effector cells» is meant the sub-population of effector cells which carry out their functions through engaging of the Fc receptors (FcRs) which they express. Considered as effector cells are lymphocytes, monocytes, neutrophils. Natural Killer (NK) cells, eosinophils, basophils, mastocytes, dendritic cells, Langerhans cells and platelets.

Preferably, the antibody of the invention has a modified Fc region imparting thereto greater affinity for the FcRn receptor.

The FcRn receptor corresponding to the «neonatal Fc receptor» is a protein composed of a heavy chain encoded by the FcRn gene (called FCGRT in Man) and a light chain: the β2-microglobulin molecule. It can bind the Fc region of IgGs and has the characteristic of increasing the half-life of IgGs which attach thereto. FcRn can be found in different organisms including, but not limited thereto, humans, mice, rats, rabbits and monkeys.

By «affinity» is meant the ability of a ligand to attach to its receptor.

By «greater affinity for FcRn» is meant an increase in the in vivo or in vitro binding affinity for FcRn of the mutated Fc region of the invention, compared with that of the parent antibody. The capability of the mutated Fc region of the invention to bind to an FcRn receptor can be evaluated in vitro by ELISA assay, as described for example in patent application WO2010/106180. In one particular embodiment, the Fc regions used in the invention have modified, advantageously increased, affinity for the FcRn receptor. This increase in binding to FcRn translates as improved serum retention in vivo, and hence an increase in half-life.

The antibodies of the invention can be used alone or in combination, for example several antibodies having different Fc regions can be administered in combination or co-administered.

In one aspect of the invention, the modified Fc region of the antibody of the invention comprises at least one mutation compared with the Fc region of a parent antibody. The mutations concerned are not the natural variations which define the isotype of immunoglobulin, but artificial mutations, the production method generating the Fc region carrying the desired mutations(s).

Preferably, the invention relates to a composition comprising a single type of antibody comprising a mutated Fc region. In other words, the composition comprises antibody molecules of identical sequence.

By «mutation» is meant a change in at least one amino acid of the sequence of a polypeptide, in particular a change of at least one amino acid in the Fc region of the parent antibody. The antibody obtained then comprises an Fc region that is mutated compared with that of the parent antibody. Preferably, mutation is a substitution, an insertion or deletion of at least one amino acid at a particular position. The mutated Fc regions can have several mutations affecting several amino acids, preferably from two to ten.

By «substitution» is meant the replacement of an amino acid by another amino acid at a particular position in a sequence of the parent antibody. For example, substitution 434S refers to a variant antibody (or mutant), here a variant in which an amino acid at position 434 is replaced by serine. Preferably the mutation lettering used is the following: «434S» or «N434S», and indicates that the parent antibody comprises asparagine at position 434, which is replaced by serine in the variant. If there is a combination of substitutions, the preferred format is the following: «259I/315D/434Y» or «V259I/N315D/N434Y». This indicates that there are three substitutions in the variant at positions 259, 315 and 434, and that the amino acid at position 259 of the parent antibody, namely valine, is replaced by isoleucine, that the amino acid at position 315 of the parent antibody, namely asparagine, is replaced by aspartic acid and that the amino acid at position 434 of the parent antibody, namely asparagine, is replaced by tyrosine.

By «deletion of amino acids» or «deletion», is meant the removal of an amino acid at a particular position in a sequence of the parent antibody. For example, E294del or 294del designates the deletion of glutamic acid at position 294.

By «insertion of an amino acid» our «insertion» is meant the addition of an amino acid at a particular position in a sequence of the parent antibody. For example, the insertion G>235-236 indicates insertion of glycine between positions 235 and 236.

Throughout the present application, the numbering of the residues in the Fc region is that of the immunoglobulin heavy chain according to EU numbering or the equivalent in Kabat et al. (Sequences of Proteins of Immunological Interest, 5^{th} Ed. Public Health Service, National Institutes of Health, Bethesda, Md., 1991). The expression «EU numbering or the Kabat equivalent» refers to EU numbering of the residues of the IgG1, IgG2, IgG3 or IgG4 human antibodies. This is illustrated on the IMGT website (http://www.imgt.org/IMGTScientificChart/Numbering/Hu_IGHGnber.html).

Preferably, the present invention concerns an antibody in which the modified Fc region has greater affinity for the FcRn receptor than the Fc region of a parent antibody, and comprises at least two mutations, said mutations being selected from among:
(i) a modification selected from among 378V, 378T, 434Y and 434S; and
(ii) at least one modification selected from among 226G, P228L, P228R, 230S, 230T, 230L, 241L, 264E, 307P, 315D, 330V, 362R, 378V, 378T, 389T, 389K, 434Y and 434S,
   the numbering being EU numbering or the Kabat equivalent, provided that mutation (i) does not take place on the same amino acid as mutation (ii).

Preferably, the present invention relates to an antibody in which the Fc region comprises at least one combination of mutations selected from among 226G/315D/434Y, 230S/315D/434Y, 230T/315D/434Y, 230T/264E/434S, 230T/389T/434S, 241L/264E/378V, 241L/264E/434S, 250A/389K/434Y, 259I/315D/434Y, 284E/378T/396L, 264E/378V/434Y, 345D/330V/434Y, 315D/382V/434Y and 378V/383N/434Y as compared with the Fc region of said parent antibody, the numbering being EU numbering or the Kabat equivalent.

Preferably, the present invention relates to an antibody in which the Fc region comprises at least one mutation selected from among 226G, 227L, 230S, 230T, 230L, 231T, 241L, 243L, 250A, 256N, 259I, 264E, 265G, 267R, 290E, 294del, 303A, 305A, 307P, 307A, 308I, 315D, 322R, 325S, 327V, 330V, 342R, 347R, 352S, 361D, 362R, 362E, 370R, 378V, 378T, 382V, 383N, 386R, 386K, 387T, 389T, 389K, 392R, 395A, 396L, 397M, 403T, 404L, 415N, 416K, 421T, 426T, 428L, 433R, 434Y, 434S and 439Rs compared with the Fc region of said parent antibody, the numbering being EU numbering or the Kabat equivalent.

Preferably, the present invention relates to an antibody in which the Fc region comprises a combination of mutations selected from among 307A/315D/330V/382V/389T/434Y, 256N/378V/383N/434Y, 315D/330V/361D/378V/434Y, 345D/330V/361D/378V/434Y, 259I/315D/434Y, 230S/315D/428L/434Y, 241L/264E/307P/378V/433R, 250A/389K/434Y, 305A/315D/330V/395A/343Y, 264E/386R/396L/434S/439R, 315D/330V/362R/434Y, 294del/307P/434Y, 305A/315D/330V/389K/434Y, 315D/327V/330V/397M/434Y, 230T/241L/264E/265G/378V/421 T, 264E/396L/415N/434S, 227L/264E/378V/434S, 264E/378T/396L, 230T/315D/362R/426T/434Y, 226G/315D/330V/434Y, 230L/241L/243L/264E/307P/378V, 250A/315D/325S/330V/434Y, 290E/315D/342R/382V/434Y, 241L/315D/330V/392R/434Y, 241L/264E/307P/378W/434S, 230T/264E/403T/434S, 264E/378V/416K, 230T/315D/362E/434Y, 226G/315D/434Y, 226G/315D/362R/434Y, 226G/264E/347R/370R/378V/434S, 308I/315D/330V/382V/434Y, 230T/264E/378V/434S, 231T/241L/264E/378T/397M/434S, 230L/264E/378W/434S, 230T/315D/330V/386K/434Y, 226G/315D/330V/389T/434Y, 267R/307P/378V/421 T/434Y, 230S/315D/387T/434Y, 230S/264E/352S/378V/434S and 230T/303A/322R/389T/404L/434S as compared with said parent antibody, the numbering being EU numbering or Kabat equivalent.

In one preferred embodiment, the Fc regions carry a mutation selected from the group composed of one mutation at amino acid 226, 227, 228, 230, 231, 233, 234, 239, 241, 243, 246, 250, 252, 256, 259, 264, 265, 267, 269, 270, 276, 284, 285, 288, 289, 290, 291, 292, 293, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 383, 384, 385, 386, 387, 389, 390, 392, 393, 394, 395, 396, 397, 398, 399, 400, 401, 403, 404, 408, 411, 412, 414, 415, 416, 418, 419, 420, 421, 422, 424, 426, 428, 433, 434, 438, 439, 440, 443, 444, 445, 446 or 447, the numbering of the amino acids in the Fc region referring to EU numbering or the Kabat equivalent.

Some positions of amino acids in the above list—namely 226, 230, 241, 256, 259, 264, 307, 315, 330, 342, 361, 362, 378, 382, 383, 389, 396, 397, 421, 428 et 434—are preferred. In particular, the mutated Fcs having high binding affinity for FcRn may comprise at least one amino acid modification at one of said amino acid positions. Amongst these, the positions 230, 264, 307, 315, 330, 378 and 434 are preferred, further preference being given to positions 264, 315, 378 and 434.

In one particular embodiment, at least two, three, four or five mutations of amino acids can substantially improve the binding affinity for FcRn compared with the parent Fc.

In one particular embodiment, the mutations are:
(i) one or two mutations selected from the group formed of positions 226, 230, 241, 264, 307, 315, 330, 342, 362, 378, 382, 389, 396, 397, 421 and 434; preferably 230, 264, 307, 315, 330, 378 and 434, further preferably 264, 315, 378 and 434; and
(ii) at least one other, that is different, from among positions 226, 227, 228, 230, 231, 233, 234, 239, 241, 243, 246, 250, 252, 256, 259, 264, 265, 267, 269, 270, 276, 284, 285, 288, 289, 290, 291, 292, 293, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355 356, 359, 360, 361, 362, 369, 370, 371, 375, 378, 380, 382, 383, 384, 385, 386, 387, 389, 390, 392, 393, 394, 395, 396, 397, 398, 399, 400, 401, 403, 404, 408, 411, 412, 414, 415, 416, 418, 419, 420, 421, 422, 424, 426, 428, 433, 434, 438, 439, 440, 443, 444, 445, 446 and 447; preferably 226, 230, 241, 264, 307, 315, 330, 342, 362, 378, 382, 389, 396, 397, 421 and 434.

In one preferred embodiment, at least one of the positions 378 and 434 is mutated; and optionally at least one other selected from the group formed by 226, 230, 241, 264, 307, 315, 330, 342, 362, 378, 382, 389, 396, 397, 421 and 434.

Preferably, the mutations are 226G, 226Y, 227S, 227L, 228R, 228L, 230S, 230T, 230L, 230A, 230Q, 231T, 231V, 233D, 234R, 239A, 241L, 241Y, 241R, 243L, 246R, 250A, 252L, 256N, 259I, 264A, 264E, 264M, 265G, 265N, 267N, 267R, 269D, 269G, 270N, 270E, 276S, 284L, 285Y, 288R, 289I, 290R, 290E, 291S, 291Q 292W, 293del, 294del, 297D, 298G, 298N, 299M, 299A, 299K, 301C, 302A, 303A, 303I, 305A, 307P, 307A, 307N, 308I, 309P, 311R, 315D, 317R, 320T, 320E, 322R, 325S, 327V, 327T, 330V, 330T, 332V, 334E, 334R, 335A, 338R, 340E, 342R, 342E, 342K, 343S, 345Q, 345G, 347R, 350A, 352S, 354P, 355Q, 355G, 356N, 359A, 360N, 360R, 361D, 361S, 362R, 362E, 369A, 370R, 371D, 375A, 375G, 378V, 378T, 378S, 380Q, 382V, 382G, 383R, 383N, 384I, 384T, 385R, 386R, 386K, 387S, 387T, 389T, 389K, 389R, 390S, 392E, 392R, 393N, 394A, 395A, 395S, 396S, 396L, 397A, 397M, 398P, 399N, 400P, 401A, 401G, 403T, 404L, 408T, 411A, 412A, 414R, 415D, 415N, 416K, 416G, 418R, 418K, 418E, 419H, 420R, 421T, 421S, 421D, 422A, 424L, 426T, 428L, 433R, 433P, 434Y, 434S, 434H, 438R, 439R, 440R, 440N, 443R, 444F, 444P, 445S, 446A, 447N and 447E, and are also described in patent application WO2010/106180.

In another embodiment, the Fc regions comprise at least one mutation selected from the group formed by 226G, 227L, 230S, 230T, 230L, 231T, 241L, 243L, 250A, 256N, 259I, 264E, 265G, 267R, 290E, 293del, 294del, 303A, 305A, 307P, 307A, 308I, 315D, 322R, 325S, 327V, 330V, 342R, 347R, 352S, 361D, 362R, 362E 370R, 378V, 378T, 382V, 383N, 386R, 386K, 387T, 389T, 389K, 392R, 395A, 396L, 397M, 403T, 404L, 415N, 416K, 421T, 426T, 428L, 433R, 434Y, 434S and 439R; preferably 226G, 230S, 230T, 230L, 241L, 264E, 307P, 315D, 330V, 342R, 362R, 362E, 378V, 378T, 382V, 389T, 389K, 396L, 397M, 421T, 434Y and 434S.

Examples of combinations of particular mutations are given below:

226G/330V, 230L/264E, 230L/378V, 230S/315D, 230S/434Y, 230T/378V, 241L/434S, 250A434Y, 264E/378T, 305A315D, 305A/330V, 305A/434Y, 307P/434Y, 315D/389T, 330V/382V, 330V/389T, 378V/421T, 389K/434Y, 389T/434Y, 396L/434S, 230T/264E, 230T/315D, 230T/434S, 230T/434Y, 241L/307P, 264E/307P, 264E/396L, 315D/362R, 315D/382V, 362R/434Y, 378V/434Y, 382V/434Y, 226G/315D, 226G/434Y, 241L/378V, 307P/378V, 241L/264E, 378V/434S, 264E/378V, 264E/434S, 315D/330V, 330V/434Y and 315D/434Y; or

226G/315D/330V, 226G/315D/434Y, 226G/330V/434Y, 230L/264E/378V, 230T/264E/378V, 230T/264E/434S, 230S/315D/434Y, 230T/315D/434Y, 230T/389T/434S, 241L/264E/434S, 241L/264E/378V, 241L/264E/307P, 241L/307P/378V, 250A/389K/434Y, 256N/378V/434Y, 259I/315D/434Y, 264E/378T/396L, 264E/378V/416K, 294del/V307P/434Y, 264E/307P/378V, 264E/396L/434S, 264E/378V/434S, 305A/315D/330V, 305A/315D/434Y, 305A/330V/434Y, 307P/378V/434Y, 315D/330V/382V, 315D/330V/389T, 315D/378V/434Y, 315D/389T/434Y, 315D/362R/434Y, 315D/382V/434Y, 315D/330V/434Y, 330V/382V/434Y, 330V/389T/434Y and 378V/383N/434Y.

In one particularly advantageous embodiment, the Fc regions carry a combination of mutations selected from among 315D/330V/361D/378V/434Y, 230S/315D/428L/434Y, 307A/315D/330V/382V/389T/434Y, 259I/315D/434Y and 256N/378V/383N/434Y.

In another embodiment, the Fc regions carry at least one mutation selected from among:

G316D, K326E, N315D, N361H, P396L, T350A, V284L, V323I, P352S, A378V, Y436H, V266M, N421T, G385R, K326T, H435R, K447N, N434K, K334N, V397M, E283G, A378T, F423L, A431V, F423S, N325S, P343S, K290E, S375R, F405V, K322E, K340E, N389S, F243I, T307P, N389T, S442F, K248E, Y349H, N286I, T359A, S383R, K334R, T394P, V259A, T393A, P352L, Q418P, V302A, L398P, F423P, S442P, V363I, S383N, S254F, K320E, G402D, I253F, V284A, A431T, N315H, Y319H, C226Y, F405L, T393I, N434S, R255W, A287T, N286Y, A231V, K274R, V308G, K414R, M428T, E345G, F243L, P247T, Q362R, S440N, Y278H, D312G, V262A, V305A, K246R, V308I, E380G, N276S, K439Q, S267G, F423Y, A231T, K320R, L410R, K320M, V412M, T307N, T366A, P230S, Y349S, A339T, K246E, K274E, A231P, I336T, S298N, L234P, S267N, V263A, E333G, V308A, K439R, K392R, S440G, V397I, I336V, Y373D, K288E, L309P, P227S, V379A, K288R, K320T, V282A, I377T, N421S and C261R,

the numbering being EU numbering or the Kabat equivalent.

Preferably, the mutants used are selected from among

N315D/A330V/N361D/A378V/N434Y, P230S/N315D/M428L/N434Y, E294del/307P/N434Y, T307A/N315D/A330V/E382V/N389T/N434Y, V259I/N315D/N434Y and T256N/A378V/S383N/N434Y.

Preferably, the mutants of the invention are produced using a production method generating the Fc region carrying the desired mutation(s). Therefore, preferably, the antibody of the invention inhibiting at least one immune checkpoint is obtained with a method comprising at least one mutagenesis step of the Fc region of the parent antibody. This mutagenesis can be random, as explained in paragraph 1 of Example 1.

Mutagenesis can also be directed mutagenesis. Preferably, directed mutagenesis is conducted in accordance with the following protocol:

Each mutation of interest in the Fc fragment is independently inserted into an expression vector containing the anti-immune checkpoint heavy chain (containing the variable portion of the anti-immune checkpoint antibody, and the constant portion of the wild-type Fc region) via overlap extension PCR using two sets of primers adapted to integrate the targeted mutation(s) with the codon(s) encoding the desired amino acid. Advantageously, when the mutations to be inserted lie close on the Fc sequence, they are added via one same oligonucleotide. The fragments obtained with PCR are combined and the resulting fragment is PCR-amplified following standard protocols. The PCR product containing the whole heavy chain of the anti-immune checkpoint mutated on the Fc fragment, can be purified on 1% agarose gel (w/v), digested with suitable restriction enzymes and cloned in the eukaryote expression vector (such as the bicistronic vector HK-Gen EFSS) which also contains the non-modified light chain of the anti-immune checkpoint antibody under consideration.

Advantageously, the mutations carried by the Fc region of the antibody of the invention impart increased half-life thereto, and allow more efficient blockading of immune checkpoints.

Preferably, the present invention relates to an antibody having functional activity mediated by the Fc region that is modified, preferably decreased, compared with that of the parent antibody.

By a functional activity mediated by the Fc region is meant the effector functions mediated by the Fc region. Included in said functional activities mediated by the Fc region are antibody-dependent cell cytotoxicity (ADCC), complement-dependent cytotoxicity (CDC), antibody-dependent cell phagocytosis (ADCP), endocytosis activity, the secretion of cytokines or a combination of at least two of these activities. Preferably, the functional activity mediated by the Fc region under consideration in the invention is selected from among ADCC, CDC, ADCP and the combinations of at least two of these activities. This functional activity can be evaluated with methods well known in the prior art.

The functional activity mediated by the Fc region is modified, in particular decreased, compared with that of the parent antibody, by a ratio of at least 2, preferably higher than 5, preferably higher than 10, preferably higher than 15, preferably higher than 20, preferably higher than 25, preferably higher than 30.

Preferably, the antibody of the invention, which comprises a modified Fc region, is such that a mutation selected from among 294Del, 293Del and 293del/294del is introduced into the Fc region of the parent antibody, the numbering being EU numbering or the Kabat equivalent. This deletion can be the sole mutation of the Fc region, or it can be accompanied by other mutations, in particular from among those listed in the present application. Therefore, according to one particular embodiment, the antibody of the invention comprises an Fc region comprising a single mutation selected from among deletion of the amino acid at position 294 (294Del), deletion of the amino acid at position 293 (293Del), dual deletion of the amino acids 293 and 294. This single mutation leads to particular glycosylation of the Fc region, namely hypersialylation, that is particularly advantageous in respect of the half-life of the glycoproteins and inflammatory processes, without change in binding to FcRn and/or protein half-life.

According to one particular embodiment, the mutated Fc regions use in the invention carry deletion of the amino acid at position 293 or 294, and also carry one or more mutations at positions selected from the following list: 226, 230, 241, 256, 259, 264, 307, 315, 330, 342, 361, 362, 378, 382, 383, 389, 396, 397, 421, 428 and/or 434.

For example, one preferred Fc region carries the combination of mutations 307P, 434Y, in association with deletion Del294.

Preferably, the present invention relates to an antibody comprising a mutated Fc region and having functional activity, mediated by the Fc region, that is modified compared with that of a parent antibody, characterized in that said Fc region comprises at least one combination of 2 mutations, said combination being selected from among:
(i) one mutation selected from among 307N, 326E, 326T, 334N, 334R, 352L, 378V, 378T, 394P, 396L, 397M and 421T; and
(ii) at least one mutation selected from among 226Y, 227S, 230S, 231V, 234P, 243I, 243L, 246R, 246E, 247T, 248E, 253F, 254F, 255W, 259A, 261R, 262A, 263A, 266M, 267N, 267G, 274E, 274R, 276S, 278H, 282A, 283G, 284L, 286I, 286Y, 287T, 288E, 288R, 290E, 298N, 302A, 305A, 307P, 308A, 308I, 308G, 309P, 312G, 315D, 316D, 319H, 320T, 320R, 320M, 322E, 323I, 325S, 333G, 334N, 334R, 336T, 339T, 340E, 343S, 345G, 349S, 349H, 350A 352S, 359A, 361H, 362R, 363I, 366A, 373D, 375R, 377T, 378V, 378T, 379A, 380G, 383R, 385R, 389S, 389T, 392R, 393A, 393I, 394P, 396L, 397I, 397M, 398P, 405V, 405L, 410R, 412M, 414R, 421T, 421S, 423L, 423Y, 423S, 423P, 428T, 431V, 431T, 434K, 434S, 435R, 436H, 439R, 440G, 440N, 442F, 442P and 447N,

the numbering being EU numbering or the Kabat equivalent, and provided that mutation (i) does not take place on the same amino acid as mutation (ii).

Preferably, the mutated Fc regions comprise at least one combination of 3 mutations, said combination comprising:

(i) one mutation selected from among 326E, 326T, 352L, 378V, 378T, 396L, 397M, 421T, 334N, 334R, 307N and 394P; and

(ii) at least 2 mutations selected from among 226Y, 227S, 230S, 231V, 234P, 243I, 243L, 246R, 246E, 247T, 248E, 253F, 254F, 255W, 259A, 261R, 262A, 263A, 266M, 267N, 267G, 274E, 274R, 276S, 278H, 282A, 283G, 284L, 286I, 286Y, 287T, 288E, 288R, 290E, 298N, 302A, 305A, 307P, 308A, 308I, 308G, 309P, 312G, 315D, 316D, 319H, 320T, 320R, 320M, 322E, 323I, 325S, 333G, 334N, 334R, 336T, 339T, 340E, 343S, 345G, 349S, 349H, 350A 352S, 359A, 361H, 362R, 363I, 366A, 373D, 375R, 377T, 378V, 378T, 379A, 380G, 383R, 385R, 389S, 389T, 392R, 393A, 393I, 394P, 396L, 397I, 397M, 398P, 405V, 405L, 410R, 412M, 414R, 421T, 421S, 423L, 423Y, 423S, 423P, 428T, 431V, 431T, 434K, 434S, 435R, 436H, 439R, 440G, 440N, 442F, 442P et 447N,

the numbering being EU numbering or the Kabat equivalent, and provided that mutation (i) does not take place on the same amino acid as mutation (ii).

Advantageously, the mutations carried by the Fc region of the modified antibody modulate the functional activity thereof, thereby allowing improved efficiency of the antibody described in the present invention.

The mutated Fc region of the antibody of the invention has modified affinity for at least one of the receptors of the Fc region (FcR) selected from among complement C1q and the receptors FcgRIIIa (CD16a), FcgRIIa (CD32a) and FcgRIIb (CD32b).

Preferably, the affinity of the mutated Fc region for complement C1q, for receptors FcgRIIIa (CD16a) and FcgRIIa (CD32a) is decreased, and the affinity of the mutated Fc region for FcgRIIIb (CD32b) is increased.

By «modified affinity for at least one of the receptors of the Fc region (FcR)» is meant an increase or decrease in the binding affinity, in vivo or in vitro, of the mutated Fc region of the invention for at least one of the molecules C1q, FcgRIIIa (CD16a), FcgRIIa (CD32a) and FcgRIIb (CD32b).

The receptors of the Fc region concerned by the present invention are the human receptors:
C1q which is involved in CDC activity,
the FcgRIIIa receptor (CD16a) involved in ADCC and has V/F polymorphism at position 158,
the FcgRIIa receptor (CD32a) involved in platelet activation and phagocytosis, it has H/R polymorphism at position 131, and
the FcgRIIb receptor (CD32b) involved in the inhibition of cell activity.

The affinity of an antibody for an FcR receptor can be evaluated with methods well-known in the prior art. For example, persons skilled in the art can determine affinity (Kd) using surface plasma resonance (SPR). Alternatively, skilled persons can conduct a suitable ELISA assay. Suitable ELISA assay allows a comparison between the binding forces of the parent Fc and mutated Fc. The detected signals specific to the mutated Fc and parent Fc are compared.

Preferably, the mutated Fc region of the antibody of the invention has increased affinity for the FcgRIIb receptor (CD32b), and comprises at least one combination of 2 mutations, said combination comprising:
i) one mutation selected from among 326E, 326T, 378V, 397M, 352L, 394P, 396L and 421T; and
ii) at least one mutation selected from among 316D, 334R, 248E, 334N, 418P, 231V, 320E, 402D, 359A, 383R, 421T and 361H,
the numbering being EU numbering or the Kabat equivalent, and provided that mutation (i) does not take place on the same amino acid as mutation (ii).

More preferably, said mutated Fc region comprises at least one combination of 3 mutations, said combination comprising:
(i) one mutation selected from among 326E, 326T, 352L, 378V, 378T, 396L, 397M, 421T, 334N, 334R, 307N and 394P; and
(ii) at least 2 mutations selected from among 226Y, 227S, 230S, 231V, 234P, 243I, 243L, 246R, 246E, 247T, 248E, 253F, 254F, 255W, 259A, 261R, 262A, 263A, 266M, 267N, 267G, 274E, 274R, 276S, 278H, 282A, 283G, 284L, 286I, 286Y, 287T, 288E, 288R, 290E, 298N, 302A, 305A, 307P, 308A, 308I, 308G, 309P, 312G, 315D, 316D, 319H, 320T, 320R, 320M, 322E, 323I, 325S, 333G, 334N, 334R, 336T, 339T, 340E, 343S, 345G, 349S, 349H, 350A 352S, 359A, 361H, 362R, 363I, 366A, 373D, 375R, 377T, 378V, 378T, 379A, 380G, 383R, 385R, 389S, 389T, 392R, 393A, 393I, 394P, 396L, 397I, 397M, 398P, 405V, 405L, 410R, 412M, 414R, 421T, 421S, 423L, 423Y, 423S, 423P, 428T, 431V, 431T, 434K, 434S, 435R, 436H, 439R, 440G, 440N, 442F, 442P, et 447N, the numbering being EU numbering or the Kabat equivalent, and provided that mutation (i) does not take place on the same amino acid as mutation (ii).

Preferably, said mutated Fc region comprises a combination of 4 mutations, said combination comprising at least one mutation (i), and at least 3 mutations (ii), and preferably a combination of 5 mutations, said combination comprising at least one mutation (i), and at least 4 mutations (ii).

Advantageously, the mutations carried by the Fc region of the modified antibody increases the affinity thereof for the inhibitory receptor FcgRIIb (CD32b) and decreases its affinity for the receptors C1q, FcgRIIIa (CD16a) and FcgRIIa (CD32a), thereby allowing its functional activity to be reduced. This therefore allows improved efficiency against immune checkpoints and a reduction in any toxic lysis effect on the effector cells of the immune system recognized by the antibodies of the invention.

The present invention preferably relates to an antibody of which the parent antibody comprises a parent Fc region which is a human Fc region, preferably an Fc region of a human IgG1 or human IgG2 or human IgG4.

The invention relates to a pharmaceutical composition comprising (i) at least one antibody and (ii) at least one pharmaceutically acceptable excipient.

By «pharmaceutical composition» is meant a composition having curative or preventive properties in respect of human or animal diseases.

In one embodiment, the pharmaceutical composition comprises a single antibody.

By «comprises a single antibody» is meant the presence of a set of antibodies all directed against the same target and having exactly the same structure at the Fc region.

In another embodiment, the pharmaceutical composition comprises two combined antibodies directed against different receptors.

By «comprises two antibodies» is meant the present of antibodies directed against two different targets, and the Fc regions thereof having structures that may differ depending on the target of the antibody.

In one particular embodiment, an antibody directed against CTLA4 is combined with an antibody directed against PD1. In another particular embodiment, an antibody directed against CTLA4 is combined with an antibody directed against TIM3. In another particular embodiment, an anti-CTLA4 antibody is combined with an antibody directed against LAG3. In another particular embodiment, an antibody directed against CTLA4 is combined with an antibody directed against KIR. In another particular embodiment, an antibody directed against CTLA4 is combined with an antibody directed against BTLA1. In another particular embodiment, an antibody directed against CTLA4 is combined with an antibody directed against a2AR. In another embodiment, an antibody directed against PD1 is combined with an antibody directed against TIM3. In another particular embodiment, an antibody directed against PD1 is combined with an antibody directed against LAG3. In another particular embodiment, an antibody directed against PD1 is combined with an antibody directed against KIR. In another particular embodiment, an antibody directed against PD1 is combined with an antibody directed against BTLA1. In another particular embodiment an antibody directed against PD1 is combined with an antibody directed against a2AR. In another particular embodiment, an antibody directed against TIM3 is combined with an antibody directed against LAG3. In another particular embodiment, an antibody directed against TIM3 is combined with an antibody directed against KIR. In another particular embodiment, an antibody directed against TIM3 is combined with an antibody directed against BTLA1. In another particular embodiment, an antibody directed against TIM3 is combined with an antibody directed against a2AR. In another particular embodiment, an antibody directed against LAG3 is combined with an antibody directed against KIR. In another particular embodiment, an antibody directed against LAG3 is combined with an antibody directed against BTLA1. In another particular embodiment, an antibody directed against LAG3 is combined with an antibody directed against a2AR. In another particular embodiment an antibody directed against KIR is combined with an antibody directed against BTLA1. In another particular embodiment, an antibody directed against KIR is combined with an antibody directed against a2AR. In another particular embodiment, an antibody directed against a2AR is combined with an antibody directed against BTLA1.

Any administration route is envisaged, in particular parenteral routes such as intravenous, intramuscular, subcutaneous, intradermal, topical or mucosal (e.g. inhalation) routes. Enteral routes (oral, rectal) and intrathecal are also possible. Preferably, the intravenous route is used.

The antibodies of the invention are generally formulated in pharmaceutical compositions comprising pharmaceutically acceptable excipients.

The pharmaceutical compositions can be in any adapted galenic form depending on the chosen administration route.

The pharmaceutical compositions that can be used in the invention advantageously comprise one or more pharmaceutically acceptable excipients or vehicles. For example, mention can be made of saline, physiological, isotonic, buffered solutions, etc., compatible with pharmaceutical use and known to skilled persons. The compositions may contain one or more agents or vehicles selected from among dispersants, solubilizers, stabilizers, preserving agents, etc. Some agents or vehicles that can be used in formulations (liquids and/or injectables and/or solids) are in particular methylcellulose, hydroxymethylcellulose, carboxymethylcellulose, polysorbate 80, mannitol, gelatine, lactose, vegetable oils, acacia, etc. The compositions can optionally be formulated in galenic forms or devices ensuring extended and/or delayed release. For this type of formulation, advantageous use is made of an agent such as cellulose, carbonates or starches.

Indications

The immune system normally recognizes tumour cells as foreign elements, and consequently brings about an anti-tumoral response. However, it happens that cancer cells set up escape strategies from the immune system; they are then no longer recognized or eliminated by the immune system. The immune checkpoints expressed on the surface of immune effector cells form a major pathway for tumour escape. The function of these molecules, when they are inhibitory (as is the case for PD1, CTLA4, LAG3, TIM3, KIR, BTLA1 and a2AR), is to attenuate immune response when they are engaged with their ligands which are often expressed by tumour cells.

The present invention more particularly relates to a pharmaceutical composition for use in the treatment of cancers. More particularly, the cancers concerned use the tumour escape pathway described above.

By «cancer» is meant any physiological condition characterized by abnormal cell proliferation.

The pharmaceutical composition of the invention is therefore used to treat different types of cancers. Examples of cancers particularly include non-small cell lung cancer (NSCLCs), non-resectable or metastatic melanomas, advanced renal cell carcinoma, bladder cancer, renal cancer, melanomas, lung cancer, lymphomas, mesothelioma, colorectal cancer, metastatic colorectal cancer, breast cancer, gastric cancer, head and neck cancer, brain tumours, glioblastomas, solid tumours, endometrial cancer, oesophageal cancer, gastric adenocarcinoma, germ cell tumours, testicular cancer, hepatocellular carcinoma, thymus cancer, diffuse large B-cell lymphomas (DLBCL), hematologic cancer, advanced hematologic cancer (such as non-Hodgkin's lymphomas, Hodgkin's lymphomas, chronic lymphoid leukaemia, multiple melanomas, acute myeloid leukaemia), astrocytomas, uveal melanomas, solid sarcomas, epithelial ovarian cancer, primary peritoneal cancer, Fallopian tube cancer, cervical cancer, anal cancer, ovarian cancer, urogenital cancer, urothelial cancer, genitourinary cancer, urogenital neoplasms, chest wall tumours, adrenocortical carcinoma, bile duct cancer, follicular lymphoma, pancreatic cancer, prostate cancer, brain metastases, liver cancers, cervical adenocarcinoma, gastrointestinal stromal tumours, metastatic brain cancer, Merkel cell carcinoma, synovial sarcoma, this list not being exhaustive.

In one embodiment, the optimized anti-PD1 antibody of the invention is used in the treatment of cancers. When a tumour antigen has already been presented to a lymphocyte via an Antigen Presenting Cell (APC), said lymphocyte acquires memory of the antigen. When later the memory lymphocyte encounters the tumour antigen in lymph node metastasis, its activation is inhibited by engaging of PD1 (expressed on the surface of the lymphocytes) with PDL1 (expressed on the surface of the tumour cells). The cancer cells are therefore able to escape from immune response. The blocking of PD1 with an anti-PD1 antibody leads to re-activation of the lymphocytes present in the tumour regions.

In one particular embodiment, the optimized anti-PD1 antibody is used in the treatment of lung cancer. In one particular embodiment, the optimized anti-PD1 antibody is used in the treatment of non-small cell lung cancer (NSCLC). In one particular embodiment, the optimized anti-PD1 antibody is used in the treatment of non-resectable or metastatic melanomas. In one particular embodiment, the optimized anti-PD1 antibody is used in the treatment of advanced renal cell carcinoma. In one particular embodiment, the optimized anti-PD1 antibody is used in the treatment of bladder cancer. In one particular embodiment, the optimized anti-PD1 antibody is used in the treatment of lymphomas. In one particular embodiment, the optimized anti-PD1 antibody is used in the treatment of mesothelioma. In one particular embodiment, the optimized anti-PD1 antibody is used in the treatment of colorectal cancer. In one particular embodiment, the optimized anti-PD1 antibody is used in the treatment of metastatic colorectal cancer. In one particular embodiment, the optimized anti-PD1 antibody is used in the treatment of breast cancer. In one particular embodiment, the optimized anti-PD1 antibody is used in the treatment of gastric cancer. In one particular embodiment, the optimized anti-PD1 antibody is used in the treatment of head and neck cancer. In one particular embodiment, the optimized anti-PD1 antibody is used in the treatment of brain tumours. In one particular embodiment, the optimized anti-PD1 antibody is used in the treatment of glioblastomas. In one particular embodiment, the optimized anti-PD1 antibody is used in the treatment of solid tumours. In one particular embodiment, the optimized anti-PD1 antibody is used in the treatment of endometrial cancer. In one particular embodiment, the optimized anti-PD1 antibody is used in the treatment of oesophageal cancer. In one particular embodiment, the optimized anti-PD1 antibody is used in the treatment of germ cell tumours. In one particular embodiment, the optimized anti-PD1 antibody is used in the treatment of testicular cancer. In one particular embodiment, the optimized anti-PD1 antibody is used in the treatment of hepatocellular carcinoma. In one particular embodiment, the optimized anti-PD1 antibody is used in the treatment of melanomas. In one particular embodiment, the optimized anti-PD1 antibody is used in the treatment of thymus cancer. In one particular embodiment, the optimized anti-PD1 antibody is used in the treatment of diffuse large B-cell lymphoma (DLBCL). In one particular embodiment, the optimized anti-PD1 antibody is used in the treatment of hematologic cancers. In one particular embodiment, the optimized anti-PD1 antibody is used in the treatment of advanced hematologic cancers such as non-Hodgkin's lymphoma, Hodgkin's lymphoma, chronic lymphoid leukaemia, multiple melanomas, acute myeloid leukaemia. In one particular embodiment, the optimized anti-PD1 antibody is used in the treatment of follicular lymphomas. In one particular embodiment, the optimized anti-PD1 antibody is used in the treatment of astrocytomas. In one particular embodiment, the optimized anti-PD1 antibody is used in the treatment of uveal melanomas. In one particular embodiment, the optimized anti-PD1 antibody is used in the treatment of solid sarcomas. In one particular embodiment, the optimized anti-PD1 antibody is used in the treatment of cancers of epithelial ovarian cancer. In one particular embodiment, the optimized anti-PD1 antibody is used in the treatment of primary peritoneal cancer. In one particular embodiment, the optimized anti-PD1 antibody is used in the treatment of Fallopian tube cancer. In one particular embodiment, the optimized anti-PD1 antibody is used in the treatment of cervical cancer. In one particular embodiment, the optimized anti-PD1 antibody is used in the treatment of anal cancer. In one particular embodiment, the optimized anti-PD1 antibody is used in the treatment of ovarian cancer. In one particular embodiment, the optimized anti-PD1 antibody is used in the treatment of urogenital cancer. In one particular embodiment, the optimized anti-PD1 antibody is used in the treatment of urothelial cancer. In one particular embodiment, the optimized anti-PD1 antibody is used in the treatment of genitourinary cancer. In one particular embodiment, the optimized anti-PD1 antibody is used in the treatment of urogenital neoplasms. In one particular embodiment, the optimized anti-PD1 antibody is used in the treatment of chest wall tumours. In one particular embodiment, the optimized anti-PD1 antibody is used in the treatment of adrenocortical carcinoma. In one particular embodiment, the optimized anti-PD1 antibody is used in the treatment of bile duct cancer. In one particular embodiment, the optimized anti-PD1 antibody is used in the treatment of fibrosarcoma.

In another embodiment, the optimized anti-CTLA4 antibody of the invention is used in the treatment of cancers. The function of the CTLA4 receptor is to inactivate the lymphocytes after presentation of their antigen by the Antigen Presenting Cells (APCs) to modulate immune response. Therefore, the antigens of cancer cells are brought by the APCs to the lymph nodes and presented to the circulating naive lymphocytes. When the naive lymphocyte encounters the antigen in the T-dependent region of the lymph node, it is activated and secretes cytokines whilst acquiring memory of the antigen. CTLA4 occurs on the surface of the lymphocyte 24 to 48 hours after encountering the tumour antigen. By attaching itself to B7-1 and B7-2 (molecules expressed on the APC), it induces lymphocyte inactivation. The cancer cells are therefore able to evade immune response. Blocking of CTLA4 by an anti-CTLA4 antibody leads to non-specific activation of the immune system by curbing physiological inhibition.

In one particular embodiment, the optimized anti-CTLA4 antibody is used in the treatment of metastatic or non-resectable melanomas. In one particular embodiment, the optimized anti-CTLA4 antibody is used in the treatment of melanomas. In one particular embodiment, the optimized anti-CTLA4 antibody is used in the treatment of renal carcinoma. In one particular embodiment, the optimized anti-CTLA4 antibody is used in the treatment of non-small cell lung cancer (NSCLC). In one particular embodiment, the optimized anti-CTLA4 antibody is used in the treatment of lung cancer. In one particular embodiment, the optimized anti-CTLA4 antibody is used in the treatment of myeloma. In one particular embodiment, the optimized anti-CTLA4 antibody is used in the treatment of lymphomas. In one particular embodiment, the optimized anti-CTLA4 antibody is used in the treatment of hepatocellular carcinoma. In one particular embodiment, the optimized anti-CTLA4 antibody is used in the treatment of breast cancer. In one particular embodiment, the optimized anti-CTLA4 antibody is used in the treatment of pancreatic cancer. In one particular embodiment, the optimized anti-CTLA4 antibody is used in the treatment of prostate cancer. In one particular embodiment, the optimized anti-CTLA4 antibody is used in the treatment brain metastasis. In one particular embodiment, the optimized anti-CTLA4 antibody is used in the treatment of solid tumours. In one particular embodiment, the optimized anti-CTLA4 antibody is used in the treatment of gastric cancer. In one particular embodiment, the optimized anti-CTLA4 antibody is used in the treatment of renal cancer. In one particular embodiment, the optimized anti-CTLA4 antibody is used in the treatment of head and neck cancers. In one particular embodiment, the optimized anti-CTLA4 antibody is used in the treatment of liver cancer. In one particular embodiment, the optimized anti-CTLA4 antibody is used in the treatment of brain cancer. In one particular embodiment, the optimized anti-CTLA4 antibody is used in the treatment of acute myeloid leukaemia. In one particular embodiment, the optimized anti-CTLA4 antibody is used in the treatment of chronic lymphoid leukaemia. In one particular embodiment, the optimized anti-CTLA4 antibody is used in the treatment of cervical adenocarcinoma. In one particular embodiment, the optimized anti-CTLA4 antibody is used in the treatment of Hodgkin's lymphoma. In one particular embodiment, the optimized anti-CTLA4 antibody is used in the treatment of non-Hodgkin's lymphoma. In one particular embodiment, the optimized anti-CTLA4 antibody is used in the treatment of gastrointestinal stromal tumours. In one particular embodiment, the optimized anti-CTLA4 antibody is used in the treatment of metastatic brain cancer. In one particular embodiment, the optimized anti-CTLA4 antibody is used in the treatment of ovarian cancer. In one particular embodiment, the optimized anti-CTLA4 antibody is used in the treatment of follicular lymphoma. In one particular embodiment, the optimized anti-CTLA4 antibody is used in the treatment of Merkel cell carcinoma. In one particular embodiment, the optimized anti-CTLA4 antibody is used in the treatment of uveal melanomas. In one particular embodiment, the optimized anti-CTLA4 antibody is used in the treatment of synovial sarcoma. In one particular embodiment, the optimized anti-CTLA4 antibody is used in the treatment of fibrosarcoma.

The pharmaceutical composition of the present invention may comprise a combination of two antibodies.

In one particular embodiment, the composition comprising the optimized anti-CTLA4 antibody and the anti-PD1 antibody combined is used in the treatment of lung cancer. In another particular embodiment, the composition comprising the optimized anti-CTLA4 antibody and the anti-PD1 antibody combined is used in the treatment of non-small cell lung cancer (NSCLC). In another particular embodiment, the composition comprising the optimized anti-CTLA4 antibody and the anti-PD1 antibody combined is used in the treatment of renal carcinoma. In another particular embodiment, the composition comprising the optimized anti-CTLA4 antibody and the anti-PD1 antibody combined is used in the treatment of melanomas. In another particular embodiment, the composition comprising the optimized anti-CTLA4 antibody and the anti-PD1 antibody combined is used in the treatment of glioblastoma. In another particular embodiment, the composition comprising the optimized anti-CTLA4 antibody and the anti-PD1 antibody combined is used in the treatment of gliosarcoma. In another particular embodiment, the composition comprising the optimized anti-CTLA4 antibody and the anti-PD1 antibody combined is used in the treatment of uveal melanoma. In another particular embodiment, the composition comprising the optimized anti-CTLA4 antibody and the anti-PD1 antibody combined is used in the treatment of ovarian carcinoma. In another particular embodiment, the composition comprising the optimized anti-CTLA4 antibody and the anti-PD1 antibody combined is used in the treatment of Fallopian tube cancer. In another particular embodiment, the composition comprising the optimized anti-CTLA4 antibody and the anti-PD1 antibody combined is used in the treatment of primary peritoneal carcinoma. In another particular embodiment, the composition comprising the optimized anti-CTLA4 antibody and the anti-PD1 antibody combined is used in the treatment of solid tumours. In another particular embodiment, the composition comprising the optimized anti-CTLA4 antibody and the anti-PD1 antibody combined is used in the treatment of breast cancer. In another particular embodiment, the composition comprising the optimized anti-CTLA4 antibody and the anti-PD1 antibody combined is used in the treatment of non-Hodgkin's lymphoma. In another particular embodiment, the composition comprising the optimized anti-CTLA4 antibody and the anti-PD1 antibody combined is used in the treatment of Hodgkin's lymphoma. In another particular embodiment, the composition comprising the optimized anti-CTLA4 antibody and the anti-PD1 antibody combined is used in the treatment of multiple myelomas. In another particular embodiment, the composition comprising the optimized anti-CTLA4 antibody and the anti-PD1 antibody combined is used in the treatment of renal cancer. In another particular embodiment, the composition comprising the optimized anti-CTLA4 antibody and the anti-PD1 antibody combined is used in the treatment of hepatocellular carcinoma. In another particular embodiment, the composition comprising the optimized anti-CTLA4 antibody and the anti-PD1 antibody combined is used in the treatment of metastatic or non-resectable melanoma. In another particular embodiment, the composition comprising the optimized anti-CTLA4 antibody and the anti-PD1 antibody combined is used in the treatment of sarcoma. In another particular embodiment, the composition comprising the optimized anti-CTLA4 antibody and the anti-PD1 antibody combined is used in the treatment of prostate cancer. In another particular embodiment, the composition comprising the optimized anti-CTLA4 antibody and the anti-PD1 antibody combined is used in the treatment of bladder cancer.

### Example 1: Effects of Optimized Anti-PD1 IgG1 Variants on the Immune System and Tumour Growth

In this example, an IgG4 isotype antibody having anti-PD1 specificity was produced with a non-mutated Fc sequence of IgG4, such as sequence SEQ ID NO: 9, to be used as reference.

1) Production of the Antibodies

The anti-PD1 IgG1 variants were obtained following the method described in application WO 2010/106180 which uses the random MUTAGEN™ mutagenesis method (WO 02/038756). Typically, this method comprised the following steps:

A/ Construction of an Fc Bank

The human Fc gene encoding residues 226 to 447 (as per EU numbering or the Kabat equivalent and illustrated in FIG. 1) derived from the heavy chain of a human IgG1 was cloned in a suitable vector such as the phagemid vector pMG58 following standard protocols well-known to those skilled in the art.

B/ Mutagenesis

Several banks were then generated following the procedure described in WO 02/038756, which uses low-fidelity human DNA polymerases for the purpose of introducing random mutations homogeneously on the whole target sequence. More specifically, three different mutases (pol β, η and ι) were used under different conditions to create complementary mutation profiles.

C/ Expression of the Fc Banks by Phage-Display and Selection of Those Variants Having Improved Binding to the Neonatal Receptor FcRn

The Fc banks were expressed using the Phage-display technique following standard protocols, and were used for selection of the Fc fragments. Selection can conform to the protocol detailed in patent application WO 2010/106180, in particular via selection on FcRn in solid or liquid phase, after which determination of the binding characteristics of the fragments to FcRn can be performed using ELISA.

D/ Production of Variants in Whole IgG1 Form in CHO and Evaluation of the Properties Thereof

The Fc variants were prepared in whole IgG1 format in the CHO cell line with anti-PD1 specificity. The steps of production in cell culture and purification of the antibodies were performed using routine techniques in the art, for characterization thereof.

Several combinations of mutations were selected. The following combinations were selected:

TABLE 1
Anti-PD1 IgG1 mutants selected with the method described in application
WO2010/106180

| Variant | Mutations |
|---|---|
| C6A_78 | T256N/A378V/S383N/N434Y |
| T5A_74 | N315D/A330V/N361D/A378V/N434Y |
| C6A_66 | E294del/T307P/N434Y |

E/ Production of Variants in Whole IgG1 Form in YB2/0 and Evaluation of the Properties Thereof

In addition, the mutant C6A_66 (E294del/T307P/N434Y) was produced in whole IgG1 form in YB2/0 (ATCC, CRL-1662) with anti-PD1 specificity. The steps of cell culture production and purification of the antibodies were performed using routine techniques in the art, for characterization thereof.

2) Characterization of Binding to FcRn and of Antigen Binding:

The binding assays to FcRn were conducted in accordance with the methods described in application WO 2010/106180. Briefly, binding at pH6 of IgG1 or IgG4 to FcRn and to the PD1 ligand were measured using a conventional ELISA assay. For this purpose, Maxisorp Immuno Plates were coated with FcRns or PD1 antigens. Next, the solutions of reference anti-PD1 IgG4, parent anti-PD1 IgG1, of C6A_78, T5A_74 and C6A_66 IgG1 produced in CHO, and the solutions of reference anti-PD1 IgG4, parent anti-PD1 IgG1, and of C6A_66 IgG1 produced in YB2/0, were added to each well to a final concentration of 0.5 μg IgG/mL and were placed in contact with goat anti-human IgG F(ab)2-HRP at the same concentration for 2 hours at ambient temperature. The IgGs aggregated to the F(ab′)2 were then incubated under gentle agitation for 1 hour at 30° C. on ELISA plates with FcRn and PD1 saturation. The plates were visualized with TMB (Pierce) and absorbances read at 450 nm. The final results indicate that binding to FcRn of all these mutants is improved, and that binding to the PD1 antigen is not altered compared with a parent anti-PD1 IgG1 (Table 2A) or reference anti-PD IgG4 (Table 2B), independently of the cell production system.

TABLE 2A
Results of characterization of binding to FcRn and to the PD1 antigen of
selected anti-PD1 IgG1 variants produced in CHO or YB2/0

| | | Binding to | Binding to |
|---|---|---|---|
| Variant | Mutations | FcRn | PD1 |
| C6A_78 | T256N/A378V/S383N/N434Y | ++ | = |
| T5A_74 | N315D/A330V/N361D/A378V/ | ++ | = |
| | N434Y | | |
| C6A_66 | E294del/T307P/N434Y | ++ | = |

(++: increased binding to FcRn compared with the parent anti-PD1 IgG1; =: same binding affinity to the PD1 antigen compared with the parent anti-PD1 IgG1).

TABLE 2B
Results of characterization of binding to FcRn and to the PD1 antigen
of the selected anti-PD1 IgG1 variants produced in CHO or YB2/0

| | | Binding to | Binding |
|---|---|---|---|
| Variant | Mutations | FcRn | to PD1 |
| C6A_78 | T256N/A378V/S383N/N434Y | ++ | = |
| T5A_74 | N315D/A330V/N361D/A378V/N434Y | ++ | = |
| C6A_66 | E294del/T307P/N434Y | ++ | = |

(++: increased binding to FcRn compared with the reference anti-PD1 IgG4; =: same binding affinity to the PD1 antigen compared with the reference anti-PD1 IgG4).

3) Effect of Selected Anti-PD1 IgG1 Antibodies on Release of Cytokines

Human T-lymphocytes were purified from PBMC using a CD4+T-lymphocyte enrichment column (such as developed by R&D Systems). Each cell culture well contained 10*5 purified T cells and 10*4 allogenic dendritic cells for a total volume of 200 μL. The anti-PD1 antibodies (C6A_78, T5A_74, C6A_66, parent IgG1, IgG4 as reference) were added at different concentrations. Some wells were incubated with anti-CD3 antibodies (positive control) or with irrelevant antibodies (negative control). The cells were placed in culture for 5 days at 37° C. On Day 5, 100 μL of medium were taken from each well to measure the number of secreted cytokines. The levels of IFN-gamma, TNF-alpha, IL-2, IL-4, IL-6, IL-10 and IL-12 cytokines were quantified by cytometric bead array (CBA)—(BD Biosciences). The results indicate that treatments with the anti-PD1 variants lead to stronger stimulation of cytokine secretion by the cells compared to treatment with the reference anti-PD1 IgG4, irrespective of the cell production system used.

4) Anti-Tumoral Effect of Anti-PD1 IgG1s on In Vivo Models

An in vivo tumour model was used to analyse the effect of the variants of anti-PD1 antibodies on tumour growth. PD1 knock-in mice (such as proposed by Isi Innovation), male and female aged 7 to 10 weeks were divided into 6 groups and given subcutaneous injections of SA1/N fibrosarcoma cells in a proportion of 2*10⁶ cells dissolved in 200 μL DMEM on Day 0. To study the anti-tumoral activity of the antibodies, the mice were treated with PBS (vehicle) or with 10 mg/kg of an irrelevant antibody (negative control), with anti-PD1 IgG4 (reference) and with the different variants of anti-PD1 IgG1 (C6A_78, T5A_74, C6A_66) in a proportion of 200 μL per injection. These injections were given via intra-peritoneal route on Days 1, 4, 8 and 11. Each of the groups contained 10 animals and the groups consisted of: (i) a vehicle group, (ii) an irrelevant antibody negative control group, (iii) an anti-PD1 IgG4 reference group, (iv) an anti-PD1 C6A_78 Ig G1 group (v) an anti-PD1 T5A_74 IgG1 group, (vi) an anti-PD1 C6A_66 IgG1 group. The mice were observed twice a week to evaluate tumour growth for about 6 weeks. The results indicate that advantageously the mutated anti-PD1s of the invention lead to an inhibitory effect of tumour growth which lasts longer than with the parent anti-PD1s but also than with the reference anti-PD1 IgG4s. This effect was able to be observed with the mutated anti-PD1 IgG1s of the invention produced in YB2/0 and with the mutated anti-PD1 IgG1s of the invention produced in CHO.

### Example 2: Effects of Optimized Anti-CTLA4 IgG1 Variants on the Immune System and Tumour Growth

1) Production of Antibodies

The anti-CTLA4 IgG1 variants were obtained with the method described in application WO 2010/106180 which uses the random mutagenesis method MUTAGEN™ (WO 02/038756). Typically, this method comprises the following steps:

A/ Construction of an Fc Bank

The human Fc gene encoding residues 226 to 447 (as per EU numbering or the Kabat equivalent and illustrated in FIG. 1) derived from the heavy chain of a human IgG1 was cloned in a suitable vector such as the phagemid vector pMG58 following standard protocols well known to persons skilled in the art.

B/ Mutagenesis

Several banks were then generated following the procedure described in WO 02/03875 which uses low-fidelity human DNA polymerases for the purpose of introducing random mutations homogeneously on the whole target sequence. More specifically, three different mutases (pol β, η and ι) were used under different conditions to create complementary mutation profiles.

C/ Expression of the Fc Banks by Phage-Display and Selection of Those Variants Having Improved Binding to the Neonatal Receptor FcRn

The Fc banks were expressed using the Phage-display technique in accordance with standard protocols, and used to select Fc fragments. Selection can conform to the protocol detailed in patent application WO 2010/106180, and in particular via selection on FcRn in solid or liquid phase, determination of the binding characteristics of the fragments to FcRn can be carried out using ELISA.

D/ Production of Variants in Whole Ig Form and Evaluation of the Properties Thereof

The Fc variants were prepared in whole IgG1 format in the YB2/0 cell line (ATCC, CRL-1662), and in CHO, with anti-CTLA4 specificity. The steps of cell culture production and purification of the antibodies were performed using routine techniques in the art, for characterization thereof.

Several combinations of mutations were selected. The following combinations were selected:

TABLE 3
Anti-CTLA4 IgG1 mutants selected with the method described in
application WO2010/106180

| Variant | Mutations |
|---|---|
| Del294 | E294del |
| C6A_66 | E294del/T307P/N434Y |

2) Characterization of Binding to FcRn and of Antigen Binding:

Binding assays to FcRn were conducted with the methods described in application WO 2010/106180. Briefly, binding of the IgG1s to FcRn at pH 6 and to the ligand CTLA4 was measured using a conventional ELISA assay. For this purpose, Maxisorp Immuno Plates were coated with FcRn or CTLA4 antigens. The solutions of parent anti-CTLA4 IgG1, Del294 and C6_A66 produced in CHO or YB2/0 were added to each well to a final concentration of 0.5 μg IgG/mL and were placed in contact with goat anti-human IgG F(ab)-HRP at the same concentration for 2 hours at ambient temperature. The IgGs aggregated to the F(ab′)2 were then incubated under gentle agitation for 1 hour at 30° C. on ELISA plates with FcRn and CTLA4 saturation. The plates were visualized with TMB (Pierce) and the absorbances read at 450 nm. The final results indicated that binding to FcRn of the Del294 mutant is unchanged and that binding of the C6A_66 mutant is increased, irrespective of the cell production system used. Binding to the CTLA4 antigen of both mutants was unchanged compared with a reference parent anti-CTLA4 IgG1 (Table 4).

TABLE 4
Results of characterization of binding to FcRn and to the CTLA4
antigen of the selected anti-CTLA4 IgG1 variants

| | | Binding to | |
|---|---|---|---|
| Variant | Mutations | FcRn | Binding to CTLA4 |
| Del294 | E294del | = | = |
| C6A_66 | E294del/T307P/N434Y | ++ | = |

(++: increased binding to FcRn compared with the parent anti-CTLA4 IgG1; =: same binding affinity to the CTLA4 antigen compared with the parent anti-CTLA4 IgG1).

3) Assay of ADCC Activity of the Anti-CTLA4 Mutants

NK cells were incubated with target eukaryote cells expressing CTLA4, in the presence of different concentrations (0.005 to 5000 ng/ml) of parent anti-CTLA4 Ig G1 and of anti-CTLA4 IgG1 variants 294del and C6A_66 produced in YB2/0 or in CHO. The level of intracellular lactate dehydrogenase (LDH) released by the lysed target cells was measured.

Human NK cells were purified from peripheral blood from healthy donors using the negative depletion technique developed by Miltenyi. The ADCC assay comprised incubation of NK cells with target eukaryote cells expressing the CTLA4 antigen, in the presence of different concentrations of anti-CTLA4 antibodies. After an incubation time of 16 hours, the cytotoxicity induced by the anti-CTLA4 antibodies was measured by quantifying, in the cell supernatants, the intracellular LDH released by the lysed target cells. The results indicate that the variants of anti-CTLA4 IgG1 show reduced ADCC activity compared with the parent anti-CTLA4 IgG1, irrespective of the cell production system used.

4) Effect of the Selected Anti-CTLA4 Antibodies on the Release of Cytokines

Human T-lymphocytes were purified from PBMC using a CD4+T-lymphocyte enrichment column (such as developed by R&D Systems). Each cell culture well contained 10*5 purified T-cells and 10*4 allogenic dendritic cells for a total volume of 200 μL. The anti-CTLA4 antibodies (Del294, C6A_66, parent IgG1 as reference) were added at different concentrations. Some wells were incubated with anti-CD3 antibodies (positive control) or with irrelevant antibodies (negative control). The cells were placed in culture for 5 days at 37° C. On Day 5, 100 μL of medium were taken from each of the wells to measure the quantity of secreted cytokines. The level of IFN-gamma, TNF-alpha, IL-2, IL-4, IL-6, IL-10 and IL-12 cytokines was quantified by cytometry bead array—CBA (BD Biosciences). The results indicate that treatments with the anti-CTLA4 variants lead to stronger stimulation of cytokine secretion by the cells compared to treatment with the parent anti-CTLA4 IgG1, irrespective of the cell production system used.

5) Anti-Tumoral Effect of Anti-CTLA4 IgG1s on In Vivo Models

An in vivo tumour model was used to analyse the effects of the variants of anti-CTLA4 antibodies on tumour growth. CTLA4 knock-in mice (such as those proposed by Oncolmmune, Inc), male and female aged 7 to 10 weeks divided into 5 groups were given subcutaneous injections of SA1/N fibrosarcoma cells in a proportion of 2*10⁶ cells dissolved in 200 μL of DMEM on Day 0. To study the anti-tumoral activity of the antibodies, the mice were treated with PBS (vehicle) or with 10 mg/kg of an irrelevant antibody (negative control), with parent anti-CTLA4 IgG1 (reference) and with the different IgG1 variants (294del, C6A_66) produced in CHO or YB2/0 in the proportion of 200 μL per injection. These injections were given via intra-peritoneal route on Days 1, 4, 8 and 11. Each of the groups contained 10 animals and the groups consisted of: (i) vehicle group, (ii) irrelevant negative control group, (iii) parent anti-CTLA4 IgG1 reference group, (iv) 294del anti-CTLA4 IgG1 group and (v) C6A_66 anti-CTLA4 IgG1 group. The mice were observed twice per week to evaluate tumour growth for about 6 weeks. The results indicate that advantageously the mutated anti-CTLA4 of the invention lead to an inhibitory effect on tumour growth which lasts longer than with the parent anti-CTLA4. This effect was able to be observed with the mutated anti-CTLA4 of the invention produced in YB2/0 and with the mutated anti-CTLA4 of the invention produced in CHO.

### Example 3: Preparation and Properties of Optimized Anti-PD1 IgG1 and IgG4 Variants

In this example, an antibody of IgG4 isotype having anti-PD1 specificity was produced with a non-mutated Fc sequence of IgG4 of the invention such as sequence SEQ ID NO: 9, to be used as reference (IgG4_S228P).

1) Production of Variant IgG1 Antibodies in Whole IgG1 Form in CHO-S

The Fc variants were prepared in whole IgG1 format in the CHO-S cell line having anti-PD1 specificity.

For this purpose, the VH and VL sequences of nivolumab (SEQ ID NO: 11 and 12 respectively), or VH and VL sequences of pembrolizumab (SEQ ID NO: 13 and 14 respectively) were used as variable portions.

Each mutation of interest in the Fc fragment was inserted independently in an expression vector containing the anti-PD1 heavy chain (containing the variable portion of the nivolumab and pembrolizumab anti-PD1 antibody, and the constant portion of the wild-type Fc region) via overlap extension PCR using two sets of primers adapted to integrate the targeted mutation(s) with the codon(s) encoding the desired amino acid. The fragments obtained by PCR were combined and the resulting fragment was amplified by PCR following standard protocols. The product of PCR containing the entire heavy chain of the mutated anti-PD1 on the Fc fragment, was purified on 1% agarose gel (w/v), digested with suitable restriction enzymes and cloned in an eukaryote expression vector also containing the non-modified light chain of the anti-PD1 antibody under consideration. The steps of cell culture production and purification of the antibodies were conducted using routine techniques in the art, for characterization thereof. The selected combinations of mutations were the following:

TABLE 5
anti-PD1 IgG1 mutants produced in the invention

| Variant | Mutations |
|---|---|
| IgG1_C6A_74 | V259I/N315D/N434Y |
| nivolumab | |
| IgG1_C6A_74Del294 | V259I/Del294/N315D/N434Y |
| nivolumab | |
| IgG1_C6A_74 | V259I/N315D/N434Y |
| pembrolizumab | |
| IgG1_C6A_74Del294 | V259I/Del294/N315D/N434Y |
| pembrolizumab | |

2) Production of the Variant IgG4 Antibodies

The anti-PD1 variants of IgG4 were obtained by directed mutagenesis in the same manner as described in the foregoing.

The selected combinations of mutations were the following:

TABLE 6
Mutants of anti-PD1 IgG4 produced in the invention

| Variant | Mutations |
|---|---|
| IgG4_S228P/C6A_74 | S228P/V259I/N315D/N434Y |
| nivolumab | |
| IgG4_S228P/C6A_74 | S228P/V259I/N315D/N434Y |
| pembrolizumab | |

3) Characterization of Binding to FcRn and to the Human CD64 Receptor (FcγRI), and of Antigen Binding:

3.1. Binding to the Human PDI Receptor (Recombinant Protein e.g. the R&D System PD1-Fc Ref. 1086-PD) and to the Human CD64 Receptor (hCD64) (hFcγRI):

Maxisorp Immuno Plates were coated with the PD1 antigen (human, cynomolgus monkey or murine) in PBS (10 ng, 10 ng, 20 ng per well respectively) or the human CD64 receptor (100 ng/well) overnight at 4° C. (100 μl/well). After saturation of the plates for 2 hours in PBS buffer and 4% BSA, the solutions of IgG1 or parent anti-PD1 IgG4 or of each variant were added to each well at increasing concentrations (from 0.00625 to 0.2 μg/ml for PD1 and from 0.03125 to 1 μg/ml for hCD64) for 1 h at 37° C. and then placed in contact with goat anti-human CK IgG F(ab′)2-HRP for 1 h at 37° C. The bound IgGs were detected after TMB visualization and absorbance measured at 450 nm.

Regarding antigen binding, the results show that all the assayed nivolumab variants (IgG1_C6A_74Del294 nivolumab, IgG1_C6A_74 nivolumab and IgG4_S228P/C6A_74 nivolumab) and the references (IgG1 WT and IgG4_S228P) recognize the human PDI antigen and the cynomolgus monkey PD1 antigen, and do not recognize the murine PD1 antigen. The same results were observed with the pembrolizumab variants.

The results are given in Tables 7a and 7b:

TABLE 7a
Binding of the different mutants of nivolumab to the human, murine or
cynomolgus PD1 antigen

| | | | | OD Ratio | |
|---|---|---|---|---|---|
| | OD 450 nm at 0.2 μg/ml | | | variant/IgG1_WT | |
| | | | cynomolgus | human | cynomolgus |
| Nivolumab variants | human PD1 | murine PD1 | PD1 | PD1 | PD1 |
| IgG1_WT | 2.6128 | 0.3052 | 2.3893 | 1.00 | 1.00 |
| IgG1_C6A_74 | 2.8741 | 0.2352 | 2.2785 | 1.10 | 0.95 |
| IgG1_C6A_74Del294 | 2.7711 | 0.3102 | 2.2859 | 1.06 | 0.96 |
| IgG4_S228P | 2.7223 | 0.3922 | 2.3300 | 1.04 | 0.98 |
| IgG4_S228P/C6A_74 | 2.6978 | 0.3022 | 2.3115 | 1.03 | 0.97 |

TABLE 7b
Binding of the different mutants of pembrolizumab to the human,
murine or cynomolgus PD1 antigen.

| | | | | OD Ratio variant/ | |
|---|---|---|---|---|---|
| | OD 450 nm at 0.2 μg/ml | | | IgG1-WT | |
| Pembrolizumab | human | murine | cynomolgus | human | cynomolgus |
| variants | PD1 | PD1 | PD1 | PD1 | PD1 |
| IgG1_WT | 1.2839 | 0.0568 | 1.2484 | 1.00 | 1.00 |
| IgG1_C6A- | 1.3410 | 0.0636 | 1.2643 | 1.04 | 1.01 |
| 74Del294 | | | | | |
| IgG4_S228P | 1.3219 | 0.1049 | 1.2391 | 1.03 | 0.99 |
| IgG4_S228P/ | 1.2512 | 0.1375 | 1.2148 | 0.97 | 0.97 |
| C6A-74 | | | | | |

With regard to binding to hCD64, the nivolumab IgG4 variants (IgG4_S228P/C6A_74 nivolumab but also IgG1_C6A-74Del294 nivolumab) show weak binding, whereas IgG1_WT and IgG1_C6A_74 nivolumab show strong binding.

The results are given in Tables 8a and 8b:

TABLE 8a
Binding of the different mutants of nivolumab to hCD64

| | | OD Ratio |
|---|---|---|
| Nivolumab variants | OD 450 nm at 0.5 μg/ml | variant/IgG1_WT |
| IgG1_WT | 1.2640 | 1.00 |
| IgG1_C6A_74 | 1.2436 | 0.98 |
| IgG1_C6A_74Del294 | 0.0574 | 0.05 |
| IgG4_S228P | 0.1136 | 0.09 |
| IgG4_S228P/C6A_74 | 0.0525 | 0.04 |

TABLE 8b
Binding of the different mutants of pembrolizumab to hCD64

| | OD 450 nm at | OD Ratio |
|---|---|---|
| Pembrolizumab variants | 0.5 μg/ml | variant/IgG1_WT |
| IgG1_WT | 0.7772 | 1.00 |
| IgG1_C6A-74Del294 | 0.0858 | 0.11 |
| IgG4_S228P | 0.218 | 0.28 |
| IgG4_S228P/C6A-74 | 0.1252 | 0.16 |

3.2. Binding to the FcRn Receptor (Recombinant Protein Human FcRnα and β2-Microglobulin):

Maxisorp Immuno Plates were coated with FcRn in a phosphate buffer at pH6 (250 ng per well) overnight at 4° C. (100 μl/well). After saturation of the plates for 2 hours in pH6 phosphate buffer and 5% skimmed milk, the solutions of IgG1 or parent anti-PD1 IgG4 or of each variant were added to each well in increasing concentrations (from 0.00488 to 10 μg/ml) for 1 h at 37° C. and then placed in contact with goat anti-human CK IgG F(ab′)2-HRP for 1 h at 37° C. The bound IgGs were detected after TMB staining and absorbance measured at 450 nm.

The results show that the assayed nivolumab variants (IgG1_C6A_74Del294 nivolumab and IgG4_S228P/C6A_74 nivolumab, but also IgG1_C6A_74 nivolumab) exhibit strong binding to FcRn. The same observations were made with the pembrolizumab variants. The results are given in Tables 9a and 9b:

TABLE 9a
Binding of the different mutants of nivolumab to FcRn

| | | OD Ratio |
|---|---|---|
| Nivolumab variants | OD 450 nm at 10 μg/ml | variant/IgG1_WT |
| IgG1_WT | 0.3169 | 1.00 |
| IgG1_C6A_74 | 2.366 | 7.47 |
| IgG1_C6A_74Del294 | 2.3029 | 7.27 |
| IgG4_S228P | 0.6645 | 2.10 |
| IgG4_S228P/C6A_74 | 2.6038 | 8.22 |

TABLE 9b
Binding of the different mutants of pembrolizumab to FcRn

| | | OD Ratio |
|---|---|---|
| Pembrolizumab variants | OD 450 nm at 10 μg/ml | variant/IgG1_WT |
| IgG1_WT | 0.3789 | 1.00 |
| IgG1_C6A-74Del294 | 1.5154 | 4.00 |
| IgG4_S228P | 0.5255 | 1.39 |
| IgG4_S228P/C6A-74 | 1.4196 | 3.75 |

3.3. The IgG variants were assayed for binding thereof to several human FcγRs using ELISA. Maxisorp Immuno Plates were coated with 50 ng hCD32aH/well or 75 ng hCD16aV/well in PBS. Immobilizer nickel-chelate plates (Hisgrab Pierce) were coated with 50 ng hCD32aR/well or 100 ng hCD32b/well in PBS. After coating overnight at 4° C., the plates were washed twice with PBS/0.05% Tween-20 and saturated with PBS/4% BSA for 2 hours at 37° C. In parallel, the supernatants were diluted in PBS to a final concentration of 1 μg IgG/ml and mixed with goat anti-CK IgG F(ab)2-HRP at the same concentration for 2 hours at ambient temperature. The IgGs aggregated to the F(ab′)2 were incubated under gentle agitation for 1 hour at 30° C. on ELISA plates saturated with different dilutions of PBS. The plates were stained with TMB (Pierce) and absorbance read at 450 nm.

At each time the results showed strong binding for IgG1_WT nivolumab and IgG1_C6A_74 nivolumab, and weak binding for the other variants. The results are given in Tables 10a and 10b:

TABLE 10a
Binding of the different mutants of nivolumab to different human FcγRs.

| | hCD32aH | | hCD16aV | | hCD32aR | | hCD32b | |
|---|---|---|---|---|---|---|---|---|
| | OD | OD | OD | OD | OD | OD | OD | OD |
| Nivolumab | 450 nm at | Ratio | 450 nm at | Ratio | 450 nm at | Ratio | 450 nm at | Ratio |
| variants | 0.5 μg/ml | variant/IgG1_WT | 0.5 μg/ml | variant/IgG1_WT | 0.5 μg/ml | variant/IgG1_WT | 0.5 μg/ml | variant/IgG1_WT |
| IgG1_WT | 1.62795 | 1.00 | 0.81855 | 1.00 | 3.7489 | 1.00 | 2.0426 | 1.00 |
| IgG1_C6A- | 1.65415 | 1.02 | 1.0735 | 1.31 | 2.4275 | 0.65 | 0.4808 | 0.24 |
| 74 | | | | | | | | |
| IgG1_C6A- | 0.0798 | 0.05 | 0.1211 | 0.15 | 0.2424 | 0.06 | 0.1324 | 0.06 |
| 74Del294 | | | | | | | | |
| IgG4_S228P | 0.2107 | 0.13 | 0.1291 | 0.16 | 1.6930 | 0.45 | 1.5403 | 0.75 |
| IgG4_S228P/ | 0.3321 | 0.20 | 0.18045 | 0.22 | 1.2640 | 0.34 | 0.7280 | 0.36 |
| C6A-74 | | | | | | | | |

TABLE 10b
Binding of the different mutants of pembrolizumab to different human FcγRs.

| | hCD32aH | | hCD16aV | | hCD32aR | | hCD32b | |
|---|---|---|---|---|---|---|---|---|
| | OD | OD | OD | OD | OD | OD | OD | OD |
| | 450 nm | Ratio | 450 nm | Ratio | 450 nm | Ratio | 450 nm | Ratio |
| Pembrolizumab | at | variant/ | at | variant/ | at | variant/ | at | variant/ |
| variants | 0.5 μg/ml | IgG1_WT | 0.5 μg/ml | IgG1_WT | 0.5 μg/ml | IgG1_WT | 0.5 μg/ml | IgG1_WT |
| IgG1_WT | 0.8098 | 1.00 | 0.5833 | 1.00 | 1.8180 | 1.00 | 1.0346 | 1.00 |
| IgG1_C6A- | 0.1010 | 0.12 | 0.1155 | 0.20 | 0.0060 | 0.003 | 0.0173 | 0.02 |
| 74Del294 | | | | | | | | |
| IgG4_S228P | 0.1073 | 0.13 | 0.0843 | 0.14 | 0.4139 | 0.23 | 0.2280 | 0.22 |
| IgG4_S228P/ | 0.1387 | 0.17 | 0.1505 | 0.26 | 0.0039 | 0.002 | 0.0124 | 0.01 |
| C6A-74 | | | | | | | | |

4) Pharmacokinetic Study of the Different Variants of the Invention:

Pharmacokinetic experiments were conducted in mice expressing hFcRn and KO homozygous for an allele of the murine FcRn and heterozygous for a transgene of human FcRn (mFcRn^{-/-} hFcRnTg).

For these pharmacokinetic studies, each animal received a single intravenous injection of IgG at 5 mg/kg in the retro-orbital venous sinus, following a similar protocol to one previously described (Petkova S B, et al. Enhanced half-life of genetically engineered human IgG1 antibodies in a humanized FcRn mouse model: potential application in humorally mediated autoimmune disease. Int Immunol 2006).

In general, the half-life was calculated from the plasma concentrations measured during the elimination phase.

The half-life time could thus be obtained:
by solving of equation:

T1/2=(Ln 2×Vd)/CL, where:

Vd=distribution volume=Dose/initial plasma concentration

CL=Clearance=Dose/AUC (area under curve)
by graphic analysis by determining on the Y-axis (concentration in μg/ml) the time interval elapsed between concentration C1 and concentration C2. It is mandatory to plot this curve in semi-logarithmic scale to ensure aligning of the experimental points in this last so-called elimination phase. The investigation time of this slope must be sufficiently long to allow accurate estimation of half-life.

Once the slope of the elimination phase has been measured (kₑ or elimination rate constant), the half-life can be calculated as follows:

T1/2=Ln 2/kₑ=0.693/kₑ

In addition, the mean residence time, MRT, can be estimated. This translates the period of time the Fc polypeptide is present in the body.

MRT can be obtained as follows:

MRT=AUC/AUMC, where:

AUC=area under the zero moment curve of plasma concentrations as a function of time,

AUMC=area under the first moment curve of plasma concentrations as a function of time.

Blood samples were taken from the retro-orbital venous sinus at multiple points in time and the IgG concentrations determined by ELISA.

At each time, the results showed an increased half-life of the mutants IgG1_C64_74Del294 nivolumab and IgG4_S228P/C6A_74 nivolumab, compared with the respective references IgG1_WT and IgG4_S228P.

The results are given in Table 11:

TABLE 11
Pharmacokinetic results of the different mutants of nivolumab

| | | Cmax | AUCinf | | CL | |
|---|---|---|---|---|---|---|
| Nivolumab variants | T½ (h) | (μg/ml) | (h * μg/ml) | Vd (ml/kg) | (ml/h/kg) | MRTinf (h) |
| IgG1_WT | 118 | 72.7 | 6477 | 114 | 0.78 | 149 |
| IgG1_C6A_74Del294 | 160 | 80.4 | 11230 | 100 | 0.519 | 220 |
| IgG4_S228P | 142 | 81.1 | 10563 | 91.1 | 0.476 | 193 |
| IgG4_S228P/C6A_74 | 221 | 87.3 | 16650 | 96.1 | 0.301 | 318 |

T½ = half-life
Cmax = maximum concentration observed
AUC = area under curve extrapolated to infinity
Vd = distribution volume
CL = clearance
MRTinf = mean residence time when the concentration profile is extrapolated to infinity

5) Characterization of Cell Bindings to the Human FcRn Receptor, to Human CD64 (FcγRI), to the Human CD16aV Receptor, and Activation Assay of PD1-Expressing Jurkat Cells:

5.1. Cell Binding Assay to the Human FcRn Receptor

Binding to the human receptor FcRn (hFcRn) was evaluated on cells, with a competitive assay using Rituxan labelled with Alexa 488 fluorochrome (Rituxan A488), and Jurkat cells expressing the hFcRn receptor on their surface (Jurkat-hFcRn).

The Jurkat-hFcRn cells were incubated for 20 minutes at 4° C. with varying concentrations (500; 250; 125; 62; 31; 15; 8; 4; 2 and 0 μg/ml) of the antibodies in Tables 5 and 6 diluted in PBS at pH6, simultaneously with Rituxan-A488 used at a fixed concentration.

After washing, the attaching of Rituxan-A488 to the hFcRn receptor expressed by the Jurkat-hFcRn cells was evaluated by flow cytometry. The mean fluorescence intensity (MFI) observed was expressed as a percentage, 100% being the value obtained with Rituxan-A488 alone and 0% the value in the absence of Rituxan-A488.

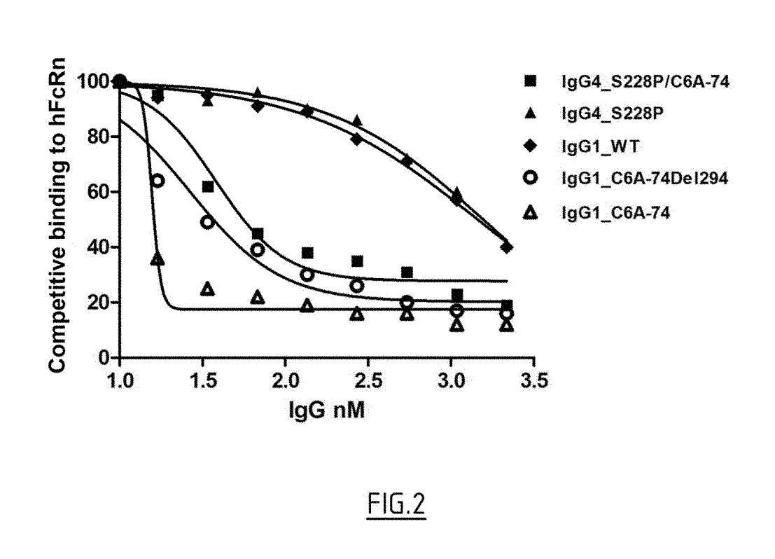

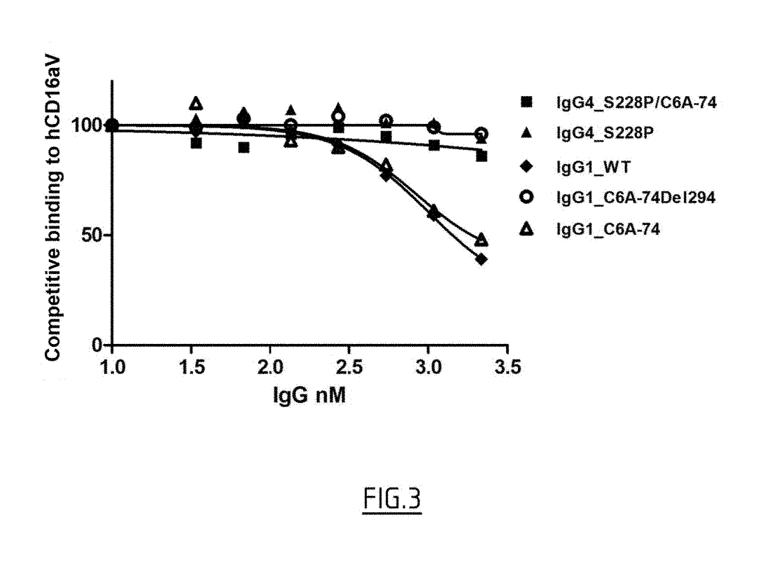

FIG. 2 shows that the nivolumab mutants of the invention bind to the hFcRn receptor, expressed on the surface of the cells, with strong affinity.

5.2. Cell Binding Assay to the Human CD64 Receptor

Binding to the human receptor CD64 was evaluated on cells with a competitive assay using Rituxan labelled with Alexa 488 fluorochrome, and Jurkat cells expressing the CD64 receptor on their surface (Jurkat-CD64).

The Jurkat-CD64 cells were incubated for 20 minutes at 4° C. with varying concentrations (500; 250; 125; 62; 31; 15; 8; 4; 2 and 0 μg/ml) of the antibodies in Tables 5 and 6 in PBS at pH7.4, simultaneously with Rituxan-A488 used at a fixed concentration.

After washing, the attaching of Rituxan-A488 to CD64 expressed by the Jurkat-CD64 cells was evaluated by flow cytometry. The mean fluorescence intensity (MFI) observed was expressed as a percentage, 100% being the value obtained with Rituxan-A488 alone and 0% the value in the absence of Rituxan-A488.

5.3. Cell Binding Assay to the Human hCD16aV Receptor

Binding to the human receptor hCD16aV was evaluated on cells with a competitive assay using Rituxan labelled with Alexa 488 fluorochrome and Jurkat cells expressing the hCD16aV receptor on their surface (Jurkat-hCD16aV).

The Jurkat-hCD16aV cells were incubated for 20 minutes at 4° C. with varying concentrations (500; 250; 125; 62; 31; 15; 8; 4; 2 and 0 μg/ml) of the antibodies in Tables 5 and 6 diluted in PBS at pH7.4, simultaneously with Rituxan-A488 used at a fixed concentration.

After washing, the attaching of Rituxan-A488 to hCD16aV expressed by the Jurkat-hCD16aV cells was evaluated by flow cytometry. The mean fluorescence intensity (MFI) observed was expressed as a percentage, 100% being the value obtained with Rituxan-A488 alone and 0% the value in the absence of Rituxan-A488.

FIG. 3 gives the results of binding of the different nivolumab mutants to the hCD16aV receptor.

5.4. Activation Assay of Jurkat-PD1 Cells Via Study of IL-2 Release

The target Jurkat cells expressing human PD1 on their surface (Jurkat-PD1) were incubated with increasing concentrations of anti-PD1 antibodies (0 to 500 ng/ml) in the presence of Jurkat cells expressing the human CD16 receptor and phorbol myristate acetate (PMA).

After an incubation time of 16 hours at 37° C., the amount of IL-2 released by the Jurkat CD16 cells was measured by colorimetry (R&D Systems DuoSet Kit IL-2).

This assay allowed verification in vitro of the absence of effector function of the antibodies produced according to the invention.

### CONCLUSION

The described assays allow confirmation of the functional properties of the different IgG1 and IgG4 variants described in the present application. In particular, the results reported allow confirmation of the advantageous properties of the IgG1 and IgG4 mutants of Nivolumab and Pembrolizumab, exhibiting an improved half-life in the body compared with the reference Igs, permitting an extended anti-tumoral effect, and having binding properties to the human Fc gamma receptors that are advantageous for improved effector properties.

1. An antibody inhibiting at least one immune checkpoint, having a modified Fc region compared with that of a parent antibody, and comprising at least two mutations, said mutations being selected from among:
(i) one mutation selected from among 378V, 378T, 434Y and 434S; and
(ii) at least one mutation selected from among 226G, P228L, P228R, 230S, 230T, 230L, 241L, 264E, 307P, 315D, 330V, 362R, 378V, 378T, 389T, 389K, 434Y and 434S,
the numbering being EU numbering or the Kabat equivalent, and provided that mutation (i) does not take place on the same amino acid as mutation (ii).

2. The antibody according to claim 1, characterized in that the immune checkpoint is an inhibitory receptor of immune effector cells.

3. The antibody according to claim 1, characterized in that the immune checkpoint is selected from among PD1, CTLA4, TIM3, LAG3, KIR, BTLA1 and a2AR

4. The antibody according to claim 1, characterized in that the Fc region comprises at least one combination of mutations selected from among 226G/315D/434Y, 230S/315D/434Y, 230T/315D/434Y, 230T/264E/434S, 230T/389T/434S, 241L/264E/378V, 241L/264E/434S, 250A/389K/434Y, 259I/315D/434Y, 284E/378T/396L, 264E/378V/434Y, 345D/330V/434Y, 315D/382V/434Y and 378V/383N/434Y compared with the Fc region of said parent antibody, the numbering being EU numbering or the Kabat equivalent.

5. The antibody according to claim 4, characterized in that the Fc region further comprises at least one mutation selected from among 226G, 227L, 230S, 230T, 230L, 231T, 241L, 243L, 250A, 256N, 259I, 264E, 265G, 267R, 290E, 294del, 303A, 305A, 307P, 307A, 308I, 315D, 322R, 325S, 327V, 330V, 342R, 347R, 352S, 361D, 362R, 362E, 370R, 378V, 378T, 382V, 383N, 386R, 386K, 387T, 389T, 389K, 392R, 395A, 396L, 397M, 403T, 404L, 415N, 416K, 421T, 426T, 428L, 433R, 434Y, 434S and 439R compared with the Fc region of said parent antibody, the numbering being EU numbering or the Kabat equivalent.

6. The antibody according to claim 1, characterized in that the Fc region comprises a combination of mutations selected from among 307A/315D/330V/382V/389T/434Y, 256N/378V/383N/434Y, 315D/330V/361D/378V/434Y, 345D/330V/361D/378V/434Y, 259I/315D/434Y, 230S/315D/428L/434Y, 241L/264E/307P/378V/433R, 250A/389K/434Y, 305A/315D/330V/395A/343Y, 264E/386R/396L/434S/439R, 315D/330V/362R/434Y, 294del/307P/434Y, 305A/315D/330V/389K/434Y, 315D/327V/330V/397M/434Y, 230T/241L/264E/265G/378V/421T, 264E/396L/415N/434S, 227L/264E/378V/434S, 264E/378T/396L, 230T/315D/362R/426T/434Y, 226G/315D/330V/434Y, 230L/241L/243L/264E/307P/378V, 250A/315D/325S/330V/434Y, 290E/315D/342R/382V/434Y, 241L/315D/330V/392R/434Y, 241L/264E/307P/378W/434S, 230T/264E/403T/434S, 264E/378V/416K, 230T/315D/362E/434Y, 226G/315D/434Y, 226G/315D/362R/434Y, 226G/264E/347R/370R/378V/434S, 308I/315D/330V/382V/434Y, 230T/264E/378V/434S, 231T/241L/264E/378T/397M/434S, 230L/264E/378W/434S, 230T/315D/330V/386K/434Y, 226G/315D/330V/389T/434Y, 267R/307P/378V/421T/434Y, 230S/315D/387T/434Y, 230S/264E/352S/378V/434S and 230T/303A/322R/389T/404L/434S compared with the Fc region of said parent antibody, the numbering being EU numbering or the Kabat equivalent.

7. The antibody according to claim 1, characterized in that the Fc region comprises a combination of mutations selected from among 315D/330V/361D/378V/434Y, 230S/315D/428L/434Y, 307A/315D/330V/382V/389T/434Y, 259I/315D/434Y and 256N/378V/383N/434Y.

8. The antibody according to claim 1, characterized in that its functional activity mediated by the Fc region is modified, in particular decreased, compared with that of the parent antibody.

9. The antibody according to claim 8, characterized in that its functional activity mediated by the Fc region is modified, in particular decreased compared with that of the parent antibody, by a ratio of at least 2, preferably higher than 5, preferably higher than 10, preferably higher than 15, preferably higher than 20, preferably higher than 25, preferably higher than 30.

10. The antibody according to claim 8, characterized in that said functional activity mediated by the Fc region is selected from among antibody-dependent cell cytotoxicity (ADCC), complement-dependent cytotoxicity (CDC), antibody-dependent cell phagocytosis (ADCP), and a combination of at least two of these activities.

11. The antibody according to claim 1, characterized in that one mutation selected from among 294Del, 293Del and 293del/294del is inserted in the Fc region of the parent antibody, the numbering being EU numbering or the Kabat equivalent.

12. The antibody according to claim 1 having functional activity, mediated by the Fc region, that is modified compared with that of the parent antibody, characterized in that said Fc region comprises at least one combination of 2 mutations, said combination being selected from among:
(i) one mutation selected from among 307N, 326E, 326T, 334N, 334R, 352L, 378V, 378T, 394P, 396L, 397M and 421T; and
(ii) at least one mutation selected from among 226Y, 227S, 230S, 231V, 234P, 243I, 243L, 246R, 246E, 247T, 248E, 253F, 254F, 255W, 259A, 261R, 262A, 263A, 266M, 267N, 267G, 274E, 274R, 276S, 278H, 282A, 283G, 284L, 286I, 286Y, 287T, 288E, 288R, 290E, 298N, 302A, 305A, 307P, 308A, 308I, 308G, 309P, 312G, 315D, 316D, 319H, 320T, 320R, 320M, 322E, 323I, 325S, 333G, 334N, 334R, 336T, 339T, 340E, 343S, 345G, 349S, 349H, 350A 352S, 359A, 361H, 362R, 363I, 366A, 373D, 375R, 377T, 378V, 378T, 379A, 380G, 383R, 385R, 389S, 389T, 392R, 393A, 393I, 394P, 396L, 397I, 397M, 398P, 405V, 405L, 410R, 412M, 414R, 421T, 421S, 423L, 423Y, 423S, 423P, 428T, 431V, 431T, 434K, 434S, 435R, 436H, 439R, 440G, 440N, 442F, 442P and 447N,
the numbering being EU numbering or the Kabat equivalent, and provided that mutation (i) does not take place on the same amino acid as mutation (ii).

13. The antibody according to claim 1, characterized in that said modified Fc region has modified affinity for at least one of the receptors of the Fc region (FcR) selected from among the complement C1q and the receptors FcgRIIIa (CD16a), FcgRIIa (CD32a) and FcgRIIb (CD32b).

14. The antibody according to claim 13, characterized in that said modified Fc region has increased affinity for the receptor FcgRIIb (CD32b), and comprises at least one combination of 2 mutations, said combination comprising:
i) one mutation selected from among 326E, 326T, 378V, 397M, 352L, 394P, 396L and 421T; and
ii) at least one mutation selected from among 316D, 334R, 248E, 334N, 418P, 231V, 320E, 402D, 359A, 383R, 421T and 361H,
the numbering being EU numbering or the Kabat equivalent, and provided that mutation (i) does not take place on the same amino acid as mutation (ii).

15. The antibody according to claim 1, characterized in that the parent antibody comprises a parent Fc region which is a human Fc region, preferably an Fc region of a human IgG1 or human IgG2 or human IgG4.

16. The antibody according to claim 15, characterized in that the parent antibody comprises a parent Fc region which is an Fc region of a human IgG4 mutated at position 228, preferably comprising the mutation S228P, preferably having the sequence SEQ ID NO: 4 or 9 and comprising the mutation S228P.

17. A pharmaceutical composition comprising (i) at least one antibody according to claim 1, and ii) at least one pharmaceutically acceptable excipient.

18. A method for treating cancer, comprising administering an antibody according to claim 1 to a patient in need thereof.

19. A method for treating cancer, comprising administering a pharmaceutical composition according to claim 17 to a patient in need thereof.


SEQUENCE LISTING (14 sequences)

SEQ ID NO: 1 - protein, 222 aa, Artificial Sequence
  Fc of IgG1 G1m1,17
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 221 aa, Artificial Sequence
  Fc of IgG2
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 222 aa, Artificial Sequence
  Fc of IgG3
CPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 222 aa, Artificial Sequence
  Fc of IgG4
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 222 aa, Artificial Sequence
  Fc of IgG1 G1m3
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Artificial Sequence
  Fc of IgG1 G1m1,17
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 228 aa, Artificial Sequence
  Fc of IgG2
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 279 aa, Artificial Sequence
  Fc of IgG3
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - protein, 229 aa, Artificial Sequence
  Fc of IgG4
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 232 aa, Artificial Sequence
  Fc of IgG1 G1m3
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 113 aa, Artificial Sequence [sequence omitted]

SEQ ID NO: 12 - protein, 107 aa, Artificial Sequence [sequence omitted]

SEQ ID NO: 13 - protein, 120 aa, Artificial Sequence [sequence omitted]

SEQ ID NO: 14 - protein, 111 aa, Artificial Sequence [sequence omitted]